(19) United States
(12) Patent Application Publication
(10) Pub. No.: US 2008/0032340 A1
(43) Pub. Date: Feb. 7, 2008

(54) Peptide motifs for binding avidin or neutravidin

(75) Inventors: Idraneel Ghosh, Tucson, AZ (US); Thomas Gaj, San Diego, CA (US); Scott Meyer, Tucson, AZ (US)

Correspondence Address: OBLON, SPIVAK, MCCLELLAND MAIER & NEUSTADT, P.C., 1940 DUKE STREET, ALEXANDRIA, VA, 22314, US

(73) Assignee: University of Arizona, Tucson, AZ (US)

(21) Appl. No.: 11/808,532

(22) Filed: Jun. 11, 2007

Related U.S. Application Data
(60) Provisional application No. 60/804,390, filed on Jun. 9, 2006.

(51) Int. Cl. C12P 21/00; B01J 13/00; C07H 21/00; C07K 1/14; C07K 7/06; G01N 33/68; G01N 33/53; C07K 7/64; C12N 1/00; C12N 15/63

(52) U.S. Cl. 435/69.1; 264/4.1; 435/243; 435/320.1; 436/501; 436/86; 530/317; 530/328; 530/329; 530/412; 536/23.1

(57) ABSTRACT

Peptide motifs DXₐAX_{b}PX_{c} (SEQ ID NO: 1) or (CDXₐAX_{b}PX_{c}CG) (SEQ ID NO: 2) that define binding to avidin or Neutravidin with high affinity, but not to streptavidin. Peptides, polypeptides and other molecules that incorporate this motif may be identified, detected, or purified by methods involving the specific binding of the motif sequence with avidin or Neutravidin. Orthogonal selection or labeling methods employing the specific binding of this peptide motif to avidin and Neutravidin, as well as utilization of the binding interaction between streptavidin and molecules that specifically bind to it.

### CROSS-REFERENCE TO RELATED APPLICATIONS

This application claims priority under 35 U.S.C. 119(e) to U.S. Provisional Application No. 60/804,390, filed Jun. 9, 2006, the entire content of which is incorporated by reference.

### STATEMENT REGARDING FEDERALLY SPONSORED RESEARCH OR DEVELOPMENT

This invention was made, in part, with funding from the National Institutes of Health under NIH grant RO1 A1068414. Therefore, the United States of America may have certain rights in the invention.

### BACKGROUND OF THE INVENTION

1. Field of the Invention

A peptide motif that defines binding to avidin or Neutravidin with high affinity, but not to streptavidin. Peptides, polypeptides and other molecules that incorporate this motif may be identified, detected, or purified by methods involving the specific binding of the motif sequence with avidin or Neutravidin. Orthogonal selection or labeling methods employing the specific binding of this peptide motif to avidin and Neutravidin, as well as utilizing the binding interaction between streptavidin and molecules that specifically bind to it.

Screening conformationally constrained peptides against macromolecular targets is of much interest in identifying novel drug leads and in developing tools for chemical biology. Specific immobilization of biotinylated macromolecular targets on avidin and streptavidin functionalized supports is often the method of choice for the selection of peptides in methodologies such as phage display, ribosome display, and mRNA display. Thus the characterization of peptide binding epitopes of avidin and streptavidin is necessary for accurate interpretation of selection and screening results. To this end, we have carried out a cyclic hexapeptide phage display selection against NeutrAvidin, a chemically deglycosylated version of avidin. The selection produced a highly homologous consensus motif (Asp-Arg/Leu-Ala-Ser/Thr-Pro-Tyr/Trp) (SEQ ID NO: 1). Two of these cyclic peptides, CDRATPYC (SEQ ID NO: 9, residues 1-8) and CDRASPY (SEQ ID NO: 7, residues 1-8), bound both NeutrAvidin™ and avidin with low μM dissociation constants whereas their acyclic counterparts bound with a significantly fold lower affinity. Moreover these peptides were found to be very specific for their targets and did not bind the structurally and functionally similar protein, streptavidin. Thus, we have identified a new class of peptides, which is distinct from the much-studied His-Pro-Gln (HPQ) motif that binds streptavidin. These results not only allow for discriminating between desired and background cyclic peptide motifs in selections and screens but also provide a new protein/peptide pair as a useful tool in chemical biology that may have utility in protein immobilization, purification, and chemical tagging. Furthermore, the widespread success of affinity tags throughout the biological sciences has prompted interest in developing new and convenient labeling strategies. Affinity tags are well-established tools for recombinant protein immobilization and purification, more recently these tags have been utilized for selective biological targeting towards multiplexed protein detection in numerous imaging applications as well as for drug-delivery. Recently, we discovered a phage-display selected cyclic peptide motif that was shown to bind selectively to NeutrAvidin and avidin but not to the structurally similar streptavidin. Here we have exploited this selectivity to develop an affinity tag based on the evolved DRATPY (SEQ ID NO: 8) moiety that is orthogonal to known Strep-tag technologies. As proof of principle the divalent AviD-tag (Avidin-Di-tag) was expressed as a Green Fluorescent Protein variant conjugate and exhibited superior immobilization and elution characteristics to the first generation Strep-tag and a monovalent DRATPY (SEQ ID NO: 8) GFP-fusion protein analogue. Additionally, we demonstrate the potential for a peptide based orthogonal labeling strategy involving our divalent AviD-tag in concert with existing streptavidin-based affinity reagents. The AviD-tag and its unique recognition properties provides researchers with a useful new affinity reagent tool for a variety of applications in the biological and chemical sciences.

2. Description of the Related Art

Labeling, detection, immobilization, and purification methods involving the binding of avidin to biotin and the binding of streptavidin to biotin are well-known. The glycoprotein avidin has an affinity for the small molecule biotin that is one of the strongest non-covalent interactions known, with a K_{d} of 10⁻¹⁵ M (Green et al., Methods. Enzymol. 184:51-67, 1990). As such, avidin, as well as the related protein streptavidin, are routinely used with biotin for immobilization in combinatorial library screenings and in vitro selections (Lin et al., Chem. Int. Ed. 41 (23):4402-4425, 2002). The (strept)avidin-biotin interactions allow for very specific immobilization, generally with low backgrounds, even from complex biological mixtures (Finn et al., Methods Enzymol. 184:244-274, 1990).

Immobilization of biomolecules is important in many drug discovery protocols, especially for peptide-based drugs. Drug discovery efforts often begin with peptide ligands as lead compounds (Gante et al., Angew. Chem. Int. Ed 33:1699-1720, 1994; Giannis et al., Angew. Chem. Int. Ed. 32:1244-167, 1993; Hruby et al., Curr. Med. Chem. 7:945-70, 2000). These peptides, in turn, are discovered in many ways, including elucidation of native ligands (Adermann, et al., Curr. Opin. Biotechnol. 15:599-606, 2004), combinatorial library screening (Lam et al., Nature 354:82-4, 1991), and in vitro selection methodologies (Smith et al., Science 228:1315-7, 1985). Selection methodologies, such as phage display (Smith et al., Chem. Rev. 97:391-410, 1997; Kehoe et al., Chem. Rev. 105:4056, 2005), ribosome display (Hanes, et al., PNAS 94:4937-42, 1997), and mRNA display (PNAS 94:12297-302, 1997), rely on immobilization of target proteins on solid surfaces that are amenable to panning procedures. One such immobilization method is the use of known biological interactions, such as the interactions between avidin and biotin or streptavidin and biotin.

One of the important methodologies to which biotin-binding proteins have been applied is phage display (Smith et al., Chem. Rev. 97:391-410, 1997). In phage display, avidin and streptavidin have been used for both direct immobilization and solution phase capture of targets (Barbas, et al., A Laboratory Manual, Cold Spring Harbor Laboratory Press, NY, 2001). A number of groups have also used streptavidin, not for immobilization, but rather as a target itself (Devlin, et al., Science 249: 404-6, 1990; Kay, et al., Gene 128:59-65, 1993; McLafferty, et al., Gene 128:29-36, 1993; Petrenko et al., Protein Eng. 13:589-92, 2000, Wilson, et al., PNAS 98:3750, 2001; Lamla, et al., J. Mol. Biol. 329:381-8, 2003). One of the earliest phage display selections was carried out by Delvin, et al. (Science 249: 404-6, 1990), and targeted streptavidin. While this target provided a convenient demonstration of the phage display methodology, the authors also recognized the importance of identifying peptides that bound streptavidin. Knowing streptavidin binding motifs allows for the identification of background sequences in screenings and selections that can be easily identified as off-target binders. Similar studies have been done to characterize peptides that demonstrate other off-target interactions, such as plastic-binding peptides (Adey et al., Gene 156:27-31, 1995).

Streptavidin and avidin have also been used as model receptors in library screenings and drug discovery. Since streptavidin is so well studied and accessible, it has been used as a target to demonstrate drug discovery methodologies, such as phage display (Devlin, Science 249:404-6, 1990), peptide library screening (Lam et al., Nature 354:82-4, 1991), and ligand-receptor interaction analysis (Weber et al., Biochemistry 31:9350, 1992). The differences in avidin and streptavidin also give insight into ligand specificity of receptors, since both proteins bind biotin with very high affinity yet share only 33% sequence identity (Green, et al., Methods Enzymol. 184:51-67, 1990).

Streptavidin-binding peptides containing the streptavidin-binding HPQ motif have been identified, Szostak et al., U.S. Pat. Nos. 6,841,359 and 7,138,253. It has been shown that peptides containing the ubiquitous consensus motif for streptavidin (HPQ) do not bind avidin in its native or deglycosylated state (Kay et al., Gene 128:59-65, 1993). Furthermore, streptavidin and avidin differ in their affinities for the biotin-competitive dye, HABA (4′-hydroxyazobenzene-2-carboxylic acid) by more than an order of magnitude (streptavidin K_{d}=100 μM, avidin K_{d}=7 μM at a pH of 7) (Green et al., Methods Enzymol. 18 (part 1):418-424, 1970).

Streptavidin has gained wider use than avidin both as a model receptor and in other biological applications, despite the abundance of avidin. This is because avidin, which can be readily isolated from hen egg white (Melamed, et al., Biochem. J. 89:591-9, 1963), has the unfavorable characteristic of diminished specificity due to its high isoelectric point (pI=10) and its glycosylated native state (Green et al., Methods Enzymol. 184:51-67, 1990). The oligosaccharide of glycosylated avidin has been shown to interact with lectin-like molecules and its positive charge at neutral pH facilitates electrostatic interactions with negatively charged species (Duhamel et al., Methods Enzymol. 184:201-7, 1990).

On the other hand, streptavidin is not glycosylated and has a relatively neutral isoelectric point (pI=5-6) (Green et al., id, 1990). However, streptavidin is not entirely free of non-specific interactions, exemplified by its motif Arg-Tyr-Asp, that mimics Arg-Gly-Asp, the universal recognition site in fibronectin and other adhesion molecules (Alon et al., Biochem. Biphys. Res. Comm. 170:1236-41, 1990).

Apart from this biotin-independent binding, streptavidin has the additional disadvantage of being more expensive to produce than avidin. In an effort to address these concerns, useful commercial variants of avidin, including a chemically deglycosylated form of the protein, called NeutrAvidin (Pierce), have been produced. Chemical modifications also reduce the isoelectric point of NeutrAvidin to a more neutral pH (pI=6.3). These modifications reduce non-specific interactions for NeutrAvidin (Hiller et al., Methods Enzymol. 184:64-70, 1990), while maintaining its biotin binding ability (Hiller et al., Biochem. J. 248:167-71, 1987), thereby providing an alternative to streptavidin for drug discovery and biological applications.

Such applications may employ affinity tags which for well over a decade affinity tags have enjoyed widespread use throughout biotechnology and are integral components of numerous research endeavors in the biological sciences [Hopp et al., A short polypeptide marker sequence useful for recombinant protein identification and purification, Biotechnology 6 (1988) 1204-1210; Lavallie, et al., Gene fusion expression systems in Escherichia coli, Curr. Opin. Biotechnol. 6 (1995) 501-506; Nilsson, et al., Affinity fusion strategies for detection, purification, and immobilization of recombinant proteins, Protein Expr. Purif. 11 (1997) 1-6; Waugh, Making the most of affinity tags, Trends Biotechnol. 23 (2005) 316-320]. These tags have aided tremendously in the production and purification of recombinant proteins [Baneyx, Recombinant protein expression in Escherichia coli, Curr. Opin. Biotechnol. (1999) 411-421; Davis, et al., New fusion protein systems designed to give soluble expression in Escherichia coli, Biotech. & Bioeng. 65 (1999) 382-388), as well as in the biochemical characterization and functional elucidation of proteins [Miller, et al., Use of actin filament and microtubule affinity chromatography to identify proteins that bind to the cytoskeleton, Methods Enzymol. 196 (1991) 303-319; Phizicky, et al. Microbiol. Rev. 59 (1995) 94-123). While primarily used for the single-step purification of recombinant proteins from complex mixtures, such as cellular lysates, affinity tags are emerging as useful tools for probing molecular function [Phizicky, et al., Protein-protein interactions: Methods for detection and analysis, Microbiol. Rev. 59 (1995) 94-123; Formosa, et al., Using protein affinity chromatography to probe structure of protein machines, Methods Enzymol. 208 (1991) 24-45; Zhang, et al., Normal and oncogenic p21ras proteins bind to the amino-terminal regulatory domain of c-Raf-1, Nature. 364 (1993) 308-313; Hu, et al., Interaction of phosphatidylinositol 3-kinase associated p85 with epidermal growth factor and platelet-derived growth factor receptor, Mol. Cell. Biol. 12 (1992) 981-990], and have recently been used as a convenient means of imaging proteins within live cells [Marks et al., In vivo targeting of organic calcium sensors via genetically selected peptides, Chem. Biol. 11 (2004) 347-356; Chen, et al., Site-specific labeling of cell surface proteins with biophysical probes using biotin ligase, Nat. Methods. 2 (2005) 99-104]. Less intrusive than large reporter proteins, fusion peptide bioconjugates can allow for the direct immobilization of a protein of interest against a fluorescent indicator [Jaiswai, et al., Use of quantum dots for live cell imaging, Nat. Methods 1 (2004) 73-78]. However, while fusion peptide based affinity labels provide an efficient means of targeting a protein of interest, specificity is often times sacrificed [Adams, et al., New biarsenical ligands and tetracysteine motifs for protein labeling in vitro and in vivo: Synthesis and biological applications, J. Am. Chem. Soc. 124 (2002) 6063-6076]. Consequently, there is a recognized need for development of less invasive and more convenient labeling strategies. Therefore, the inventors pursued the development of new peptide based labeling methods that permit the study of proteins in their native state, not only for the isolation and visualization of proteins under a particular set of conditions, but also for the biochemical classification of many proteins involved in essential cellular processes [Lesley, et al., High-throughput proteomics: Protein expression and purification in the postgenomic world, Protein Expr. Purif. 22 (2001) 159-164; Shih, et al., High-throughout screening of soluble recombinant proteins, Protein Sci. 11 (2002) 1714-1719].

A wide variety of affinity tags have been developed and are used throughout biotechnology. The most commonly employed affinity tags range from short polypeptide sequences [Hopp, et al., A short polypeptide marker sequence useful for recombinant protein identification and purification, Biotechnology 6 (1988) 1204-1210; Nygren, et al., Engineering proteins to facilitate bioprocessing. Trends Biotechnol. 12 (1994) 184-188; Skerra, et al., Applications of a peptide ligand for streptavidin: The Strep-tag, Biomol. Eng. 16 (1999) 79-86], to whole proteins, which can confer advantageous solubility effects [Baneyx, Recombinant protein expression in Escherichia coli, Curr. Opin. Biotechnol. 10 (1999) 411-421]. For example, the specific molecular recognition properties of complete protein domains such as glutathione S-transferase and the maltose-binding protein have been exploited for recognition of immobilized glutathione and maltose/amylose, respectively [Smith, et al., Single-step purification of polypeptides expressed in Escherichia coli as fusions with glutathione S-transferase, Gene. 67 (1998) 31-40; Kellerman, et al., Maltose-binding protein from Escherichia coli, Methods Enzymol. 90 (1982) 459-463]. In addition to these large whole proteins, small peptide epitopes such as polyhistidine tags [Janknecht, et al., Rapid and efficient purification of native histidine-tagged protein expressed by recombinant vaccinia virus, Proc. Natl. Acad. Sci. USA 88 (1991) 8972-8976], which can bind to immobilized metal chelates, as well as the myc-tag and FLAG-tag [Evan, et al., Isolation of monoclonal antibodies specific for human c-myc proto-oncogene product, Mol. Cell. Biol. 12 (1985) 3610-3616; Brizzard, et al., Immunoaffinity purification of FLAG epitope-tagged bacterial alkaline phosphatase using a novel monoclonal antibody and peptide elution, Biotechniques 16 (1994) 730-735], which can bind to immobilized antibodies, are commonly used for the isolation and immobilization of recombinant proteins.

nether small peptide epitope that has gained wide use is the streptavidin specific Strep-tag [Schmidt, et al., The random peptide library-assisted engineering of a C-terminal affinity peptide, useful for the detection and purification of a functional Ig Fv fragment, Protein Eng. 6 (1993) 109-122]. The development of streptavidin targeted fusion peptides has aided in a variety of unique biochemical applications and has made streptavidin, the non-glycosylated bacterial relative of avidin, the preferred protein in many applications of the (strept)avidin-biotin technologies [Schmidt, et al., Molecular interaction between the Strep-tag affinity peptide and its cognate target, streptavidin, J. Mol. Biol. 255 (1996) 753-766; Keefe, et al., One-step purification of recombinant proteins using a nanomolar-affinity streptavidin-binding peptide, the SBP-tag, Protein Expr. Purif. 23 (2001) 440-446; Lamla, et al., The nano-tag, a streptavidin-binding protein for the purification and detection of recombinant proteins, Protein Expr. Purif. 33 (2003) 39-47]. Having the unfavorable characteristic of reduced specificity due to its high isoelectric point (pI=10) and glycosylated native site [Green, Avidin and streptavidin, Methods Enzymol. 184 (1990) 51-67], avidin is sub-optimal for some biological applications. However, many useful commercial variants of avidin have been recently developed, including the chemically deglycosylated and neutral form of the protein [Hiller, et al., Nonglycosylated avidin, Methods Enzymol. 184 (1990) 68-70], called NeutrAvidin™ (“Neutravidin”) (Pierce). These chemical modifications have reduced non-specific interactions for NeutrAvidin while maintaining its biotin-binding ability [Duhamel, et al., Prevention of nonspecific binding of avidin, Methods Enzymol. 184 (1990) 201-207; Hiller, et al., Biotin binding to avidin. Oligosaccharide side chain not required for ligand association, Biochem. J. 248 (1987) 167-171], providing an alternative to streptavidin in many biological applications.

A new class of NeutrAvidin/avidin-binding cyclic peptides has been recently reported [Meyer et al., Highly selective cyclic peptide ligands for NeutrAvidin and avidin identified by phage display, Chem. Biol. Drug Des. 68 (2006) 3-10] (specifically incorporated by reference) that may be applied for a wide variety of applications, as demonstrated for the streptavidin-binding Strep-tag [Skerra, et al., Applications of a peptide ligand for streptavidin: The Strep-tag, Biomol. Eng. 16 (1999) 79-86].

Small peptides such as the Strep-tag can easily be expressed as fusions with larger proteins for use in purification (Schmidt et al., Molecular interaction between the Strep-tag affinity peptide and its cognate target, streptavidin. J Mol Biol 1996; 255 (5):753-66) or other conjugation applications (Skerra et al., Applications of a peptide ligand for streptavidin: the Strep-tag. Biomol Eng 1999; 16 (1-4):79-86). The availability of labeled streptavidin, as well as streptavidin immobilized on solid supports, have made these peptides extremely useful. The ability to use NeutrAvidin in similar situations provides valuable new tools for drug discovery and biological applications.

A monovalent peptide selection against avidin or NeutrAvidin has not been reported to date (Petrenko et al., Phages from landscape libraries as substitute antibodies. Protein Eng 2000; 13 (8):589-92). Thus, consensus motifs obtained from phage display selection against NeutrAvidin will help map the off-target sequences for combinatorial peptide library screening and in vitro selections like phage display. Additionally, peptides that bind NeutrAvidin can be used in bioconjugation applications similar to those of the streptavidin-binding peptide, Strep-tag (Skerra et al., Applications of a peptide ligand for streptavidin: the Strep-tag. Biomol Eng 1999; 16 (1-4):79-86).

The inventors have pursued the goals of a) providing known background motifs for in vitro selections and screenings, b) developing new reagents for NeutrAvidin technology, and c) studying ligand differentiation in model receptors, and herein disclose the results of an in vitro selection using a phage-displayed six residue disulfide constrained cyclic peptide library against NeutrAvidin. The resulting peptides' affinities for NeutrAvidin were characterized via a competition assay with the biotin-competitive dye, HABA, and the specificities of the peptides for NeutrAvidin were explored by analogous assays with avidin and streptavidin.

### BRIEF SUMMARY OF THE INVENTION

The inventors have discovered a novel Neutravidin motif that is reproducibly selected by phage display. This motif, DXₐAX_{b}PX_{c} (SEQ ID NO: 1) (where Xₐ=R or L; X_{b}=S or T; and X_{c}=Y or W) or CDXₐAX_{b}PX_{c}CG (SEQ ID NO: 2) (where Xₐ=R or L; X_{b}=S or T; and X_{c}=Y or W), has now been characterized by a competition with the biotin-competitive dye HABA and found to have binding constants between 12 μM and 63 μM for both NeutrAvidin and avidin. Furthermore, the selectivity shown for avidin and NeutrAvidin vs. streptavidin was greater than 1000-fold. The discovery of this motif permits the following aspects of the invention.

Methods for making polynucleotides, such as DNA or RNA, or modified or stabilized DNA and RNA, encoding a peptide sequence comprising DXₐAX_{b}PX_{c} (SEQ ID NO: 1) or CDXₐAX_{b}PX_{c}CG (SEQ ID NO: 2) are well-known and polynucleotide sequences encoding such peptides can easily be deduced based on the genetic code. Polynucleotides encoding specific peptides conforming to this motif, such as DLASPW (SEQ ID NO: 4), DRASPY (SEQ ID NO: 6), or DRATPY (SEQ ID NO: 8), can be produced. Such polynucleotides may be in single stranded or duplex form and may optionally be linked to other polynucleotide sequences, such as to sequences encoding a polypeptide to be tagged or immobilized. The polynucleotide sequences may be placed in vectors, such as plasmids, or in phage vectors, or in phage libraries.

Polynucleotides encoding the motif CDXₐAX_{b}PX_{c}CG) (SEQ ID NO: 2), which may be used to produce peptides which are cyclized by association of the two cysteine residues are also contemplated. These polynucleotides may encode specific polypeptides such as those comprising CDLASPWCG (SEQ ID NO: 5), CDRASPYCG (SEQ ID NO: 7), or CDRATPYCG (SEQ ID NO: 9). Polynucleotides encoding other peptides comprising the motif of SEQ ID NO: 1 which may be cyclized via residues other than cysteine or chemically cyclized are also contemplated.

The polynucleotides encoding the peptide motifs of SEQ ID NOS: 1 and 2 may also be fused or linked, e.g., via polynucleotide linkers, to polynucleotides encoding other polypeptides, such as those polynucleotides encoding exogenous polypeptides of interest. The sequence encoding the motifs may be spliced to either end of a chimeric polynucleotide sequence so as to provide a polynucleotide with one of the peptide motifs at either the C-terminal or N-terminal end. A chimeric or engineered polynucleotide may also express a polypeptide having one of the peptide motifs internally.

If desired, linkers, sequences encoding chemical or enzymatic cleavage sites may be inserted in the chimeric polynucleotide so as to permit removal of the avidin or Neutravidin binding residues.

Vectors, such as plasmids and phages, and host cells suitable for expressing the polynucleotides disclosed above are well-known and are also incorporated by reference to Current Protocols in Molecular Biology (June, 2007).

Polynucleotides encoding fusion or chimeric proteins or phage encompassing the peptide motifs of the invention may be used to engineer peptides or polypeptides that bind to avidin or Neutravidin. Such a method would involve expressing a polynucleotide encoding DXₐAX_{b}PX_{c} (SEQ ID NO: 1) or CDXₐAX_{b}PX_{c}CG (SEQ ID NO: 2) of the invention in a host cell for a time and under conditions suitable for expression of the engineered or recombinant polypeptide. Such a polypeptide could also be produced by peptide synthesis. The desired engineered polypeptide could be recovered by means of its ability to bind to avidin or NA or by other well-known polypeptide purification procedures.

The peptides and polypeptides of the invention comprise DXₐAX_{b}PX_{c} (SEQ ID NO: 1) or (CDXₐAX_{b}PX_{c}CG) (SEQ ID NO: 2). Such peptides include peptides comprising the following sequences DLASPW (SEQ ID NO: 4), DRASPY (SEQ ID NO: 6), DRATPY (SEQ ID NO: 8), CDLASPWCG (SEQ ID NO: 5), CDRASPYCG (SEQ ID NO: 7), or CDRATPYCG (SEQ ID NO: 9). The peptides of the invention may be linear, aligned or arrayed to provide a particular secondary structure, or configured in a particular tertiary conformation. For example, the subject peptides or polypeptides, or segments of them, may be cyclic or otherwise conformationally constrained, e.g., by expression in a phage or as part of a larger conformationally restrained molecule or molecular complex).

Dimers, trimers and multimers of the polypeptides comprising the motifs of SEQ ID NOS: 1 and 2 may be easily constructed by genetic engineering, chemical synthesis or by chemical means, such as chemical conjugation or linkage of monomers of theses motifs.

The polypeptides of the invention may, in addition to the motif of SEQ ID NO: 1 or 2 contain an exogenous amino acid sequence of interest and the peptide sequences of SEQ ID NO: 1 or 2 may be optionally attached to the N-terminal or C-terminal of the exogenous amino acid sequence of interest. Chemical or enzymatic cleavage sites may also appear in these polypeptides, for example, to facilitate removal of portions of the polypeptide that bind to avidin or Neutravidin. The polypeptides of the invention may also be bound to a solid support, such as a bead, membrane, or microtiter plate.

Peptides comprising SEQ ID NOS: 1 or 2 may also be employed in protein analytic techniques such as those described above, or by Current Protocols in Molecular Biology (June, 2007), especially Chapter 10 “Analysis of Proteins”.

The peptide or polypeptides of the invention containing the motifs of SEQ ID NOS: 1 and 2 may have dissociation constants for avidin or Neutravidin, or for both, of less than 10 μM, less than 100 μM, less than 500 μM, less than 100 nM, less than 10 nM.

Preferably these peptides will have little or no binding affinity for streptavidin. The lack of binding affinity for streptavidin may be reflected by polypeptides that do not contain a motif for streptavidin binding, such as the Histidine-Proline-Glutamine (HPQ), Histidine-Proline-Methionine (HPM), Histidine-Proline-Asparagine (HPN), Histidine-Glutamine-Proline (HQP) motifs or the following peptide sequences DVEAWL/I (SEQ ID NO: 10), EPDWF/Y (SEQ ID NO: 11), GDF/WXF (SEQ ID NO: 12), PWXWL (SEQ ID NO: 13), and VPEY (SEQ ID NO: 14).

The polypeptides (or polynucleotides) of the invention may also be formulated as compositions. For example, the polypeptides comprising the motifs of SEQ ID NO: 1 or 2 may be admixed with a lipid. The lipids may be phospholipids such as phosphatidylcholine (PC), phosphatidylethanolamine (PE), phosphatidylserine (PS), phosphatidylinositol (PI), or cholesterol. Micelles or lipid bilayers containing the polypeptides of the invention in either a hydrophilic or hydrophobic (e.g., in the membrane) may be constructed by those of skill in the art. Optionally, these lipids may be covalently attached to the polypeptides of the invention.

A molecule, such as a polypeptide, may be isolated or purified by making use of the binding interaction between DXaAXbPXc (SEQ ID NO: 1) or (CDXₐAX_{b}PX_{c}CG) (SEQ ID NO: 2) and avidin or Neutravidin. Such a method may involve contacting a composition comprising a protein of interest that either contains the binding motif of SEQ ID NO: 1 or 2 or contains an avidin or Neutravidin binding site for the peptide motif of SEQ ID NO: 1 or 2. For example, a polypeptide conjugated or associated with avidin or Neutravidin may be isolated by it specifically binding to a peptide having SEQ ID NO: 1 or 2. Optionally, the peptide may be bound to a bead, substrate or other support. Alternatively, a peptide encompassing SEQ ID NO: 1 or 2 may be isolated or purified by binding it to a substrate or solid support to which avidin or Neutravidin is bound. Unbound molecules in a composition containing a protein of interest may be removed, and the bound molecules recovered, for instance, by elution from the material or substrate to which they are bound. The protein to be isolated or purified may be tagged or conjugated to the peptide or polypeptide having the motif of SEQ ID NO: 1 or 2 or, alternatively, to a corresponding avidin or Neutravidin binding site.

A target molecule that binds to DXaAXbPXc (SEQ ID NO: 1) or (CDXₐAX_{b}PX_{c}CG) (SEQ ID NO: 2) may also be identified using the specific interaction of peptides comprising SEQ ID NOS: 1 or 2 and avidin and/or Neutravidin. Such a method may proceed by contacting said target molecule with a polypeptide containing SEQ ID NO: 1 or 2, which may be tagged or labeled. Such a method may be performed using conventional and well-known immunoassay, flow cytometry, or bioimaging procedures. Such a method may also involve use of cross-linked avidin or Neutravidin or multimeric or cross-linked peptides containing SEQ ID NOS: 1 or 2.

Peptides containing SEQ ID NOS: 1 and 2 that bind to avidin or Neutravidin, but not to streptavidin may be employed in an orthogonal selection method. This permits separate identification of molecules binding to streptavidin and to avidin or Neutravidin.

In selection procedures involving avidin or Neutravidin, off-target binders may be found by identifying peptides comprising DXaAXbPXc (SEQ ID NO: 1) or SEQ ID NO: 2 and identifying such peptides as off-target binders. Such a “false positive” selection improves the efficiency of high throughput screening methods and other screening assays involving avidin or Neutravidin binding by permitting the screener to eliminate samples that bind to avidin or Neutravidin but which do not participate in the binding assay of interest.

### BRIEF DESCRIPTION OF THE DRAWINGS

FIG. 1: Competitive ligand binding analysis of CDRASPYCG (SEQ ID NO: 7) (black triangles), CDRATPYCG (SEQ ID NO: 9) (gray circles) and CDLASPWCG (SEQ ID NO: 5) (empty squares) for (A) cyclized peptides with the HABA-Neutravidin complex, (B) uncyclized peptides with the HABA-Neutravidin complex, (C) cyclized peptides with the avidin-HABA complex and (D) cyclized peptides with the streptavidin-HABA complex. Protein-HABA complexes were 50 μM in concentration for (A-D). The absorbance was measured at 500 nm and normalized to the 50 μM HABA-protein complex. Error bars indicate the standard deviation of three separate assays. In cases where no error bars are seen, they are smaller than the symbols used in the figure. The best-fit lines to equation 2 in (A) and (C) are shown for CDRASPYCG (SEQ ID NO: 5) (solid black), CDRATPYCG (SEQ ID NO: 9) (solid grey), and CDLASPWCG (SEQ ID NO: 5) (dashed black).

FIG. 2: The 3D structure of (A) avidin (PDB ID: 1AVD) (Pugliese et al., Three-dimensional structure of the tetragonal crystal form of egg-white avidin in its functional complex with biotin at 2.7 A resolution. J Mol Biol 1993; 231 (3):698-710) and (B) streptavidin (PDB ID: 1STP) (Weber, et al., Structural origins of high-affinity biotin binding to streptavidin. Science 1989; 243 (4887):85-8) bound to biotin (illustrated as black spheres within the interior of each protein). Amino acids represented as light spheres are conserved residues within 12 Å of the biotin molecule. Amino acids represented as dark sticks are divergent residues within the same area. The positively charged Arg114, Lys92, and Lys45, are labeled in (A) (Arg100 is not visible). The analogous residues from streptavidin, Leu124 and Asn105, (Leu56 and Thr111 are not visible) are labeled in (B). The chemical structure of the selected peptide CDRASPYCG (SEQ ID NO: 9) is shown in (C). Alternate residues found in the consensus motif are indicated in parentheses.

FIG. 3. Ribbon representations of GFPuv and Venus fusion proteins. The NeutrAvidin/avidin specific monovalent Avi-tag and divalent AviD-tag (sequences shown in red) were expressed conjugated to the N-terminal domain of GFPuv (PDB ID: 2EMD). The streptavidin specific Strep-tag (sequence shown in blue) was expressed conjugated to the C-terminal domain of the Yellow Fluorescent Protein Venus (PDB ID: 1MYW).

FIG. 4. Immobilization of Avi-tag GFPuv and AviD-tag GFPuv fusion proteins on agarose immobilized NeutrAvidin resin. 200 pMol of tagged GFPuv and untagged GFPuv were incubated with 100 μL of NeutrAvidin resin for 1 hr at room temperature. Following incubation, NeutrAvidin resins were centrifuged, washed with 100 μL buffer and photographed under UV light for direct visualization of GFPuv fluorescence. Following the fifth wash, NeutrAvidin resins were incubated with 100 μL of a 250 μM biotin solution for 30 min. at room temperature followed by UV visualization.

FIG. 5. Affinity purification of fusion proteins from cell lysate. (A) 400 μL of agarose immobilized NeutrAvidin incubated with cell lysate containing 100 μg of Avi-tag GFPuv. (B) 400 μL of agarose immobilized NeutrAvidin incubated with cell lysate containing 100 μg of AviD-tag. (C) 400 μL of agarose immobilized streptavidin resin incubated with cell lysate containing 100 μg of Avi-tag 2 GFPuv. (A-C) Following 6 column washes, immobilized protein was eluted with 500 μM biotin. Levels of protein elution and purity were resolved on a 15% SDS-polyacrylamide gel under reducing conditions.

FIG. 6. Orthogonal binding of the AviD-tag and Strep-tag. 3 nmol of Avi-tag-GFPuv (A) and Strep-tag Venus (B) were incubated with 400 μL agarose immobilized NeutrAvidin (white) and streptavidin (black) resin. Following 1 hr. each column was washed successively with 6 buffer washes. Immobilized protein was eluted with 500 μM biotin and collected in 400 μL fractions. Fluorescence for each column wash and eluate was measured with a Molecular Devices' SpectraMax Gemini microplate spectrofluorimeter. Florescence intensity is normalized to Wash 1 for each fusion protein.

The patent or application file contains at least one drawing executed in color. Copies of this patent or patent application publication with color drawing(s) will be provided by the office upon request and payment of the necessary fee.

### DETAILED DESCRIPTION OF THE INVENTION

Previous studies on various streptavidin-selected HPQ epitopes showed that they did not bind avidin in either the glycosylated or deglycosylated state (Kay et al., An M13 phage library displaying random 38-amino-acid peptides as a source of novel sequences with affinity to selected targets. Gene 1993; 128 (1):59-65; Gregory et al., Use of a biomimetic peptide in the design of a competitive binding assay for biotin and biotin analogues. Anal Biochem 2001; 289 (1):82-8). Studies on multivalent landscape peptides selected for NeutrAvidin affinity (Petrenko et al., Phages from landscape libraries as substitute antibodies. Protein Eng 2000; 13 (8):589-92) showed no binding to streptavidin. This specificity permits the use of the two classes of peptides in mixed systems where orthogonal recognition (intermolecular interactions that operate independently of each other so that no significant crossover or interference occurs) of NeutrAvidin and streptavidin would be beneficial. Besides being an interesting off-target consensus motif for NeutrAvidin immobilized screenings and in vitro selections, this epitope presents an alternative to the HPQ sequences used in a variety of applications. For instance, a sequence based on the DXₐAX_{b}PX_{c} (SEQ ID NO: 1) motif can be used as an immobilization tag for protein purification (Schmidt et al., Molecular interaction between the Strep-tag affinity peptide and its cognate target, streptavidin. J Mol Biol 1996; 255 (5):753-66) or for immunoassays and blots (Skerra et al., Applications of a peptide ligand for streptavidin: the Strep-tag. Biomol Eng 1999; 16 (1-4):79-86). Alternately, the NeutrAvidin binding peptides may be used in a bivalent phage display system, similar to the method developed by Chen, et al. (Chen et al., Design and validation of a bifunctional ligand display system for receptor targeting. Chem Biol 2004; 11 (8):1081-91) for targeting cell receptors, or in producing cell-penetrating protein (CPP) complexes (Boonyarattanakalin et al., Synthesis of an artificial cell surface receptor that enables oligohistidine affinity tags to function as metal-dependent cell-penetrating peptides. J Am Chem Soc 2006; 128 (2):386-7).

A 6-residue cyclic peptide library, when selected to recognize NeutrAvidin, resulted in the identification of a unique motif:
DXₐAX_{b}PX_{c} (SEQ ID NO: 1)
(where Xₐ=R or L; X_{b}=S or T; and X_{c}=Y or W).

Several cyclic peptides have been individually characterized and shown to bind both NeutrAvidin and avidin with low micromolar dissociation constants, with the peptide DRATPY (SEQ ID NO: 2) binding the most tightly with a dissociation constant of 12 μM. The inventors have also demonstrated that this molecular epitope is highly selective for NeutrAvidin/avidin and does not interact with the structurally similar biotin binding protein, streptavidin. The discovery of this motif provides a NeutrAvidin/avidin specific affinity tag.

Moreover, the inventors show that recombinant proteins expressed in conjugation with two copies of this peptide sequence, which have been named the AviD-tag (Avidin-Di-tag), can be successfully immobilized onto a NeutrAvidin support, thus allowing for the single-step purification of recombinant proteins in yields greater than the original Strep-tag [Essen, et al., Single-step purification of a bacterially expressed antibody Fv fragment by immobilized metal affinity chromatography in the presence of betaine. J. Chromatogr A. 657 (1993) 55-6]. The orthogonal nature of the AviD-tag and Strep-tag permits multiplexed labeling of distinct proteins in complex biological mixtures.

Polynucleotides encoding a polypeptide containing the DXₐAX_{b}PX_{c} (SEQ ID NO: 1) motif can easily be designed based on the genetic code and, if desired, the optimization of codon usage for a particular host. Polynucleotides encoding this motif may be added to polynucleotides in a library to permit isolation of tagged recombinant proteins. Furthermore, polynucleotides encoding protease splice sites may be inserted between the motif sequence used as a tag and the nucleotides encoding a protein segment of interest.

In one aspect of the invention the inventors have discovered, through cyclic peptide phage display, novel avidin/NeutrAvidin specific motifs that bind to these proteins in a HABA-competitive manner. The newly identified epitopes identify false positives from in vitro selections, and also provide useful tools for drug discovery.

The term “polynucleotide” encompasses both DNA and RNA, as well as duplex and non-duplex polynucleotides. Polynucleotides which encode amino acid sequences may be easily constructed based on the known genetic code.

Unless otherwise specifically defined, the terms “peptide”, “polypeptide”, and “protein” are used interchangeably in this disclosure and refer to two or more peptide-bonded natural or modified D- or L-amino acid residues. Generally, a peptide or polypeptide of the invention will comprise L-amino acid residues. Peptides may sometimes be specifically defined as containing between 2 and 99 amino acid residues, e.g., 10, 20, 50, 60 or 99 residues and polypeptides may sometimes be specifically defined as containing 100 or more amino acid residues. Modified peptides or polypeptides are also contemplated, such as glyco- or lipopeptides or proteins.

The terms “avidin” and “streptavidin” encompass their conventional meanings as disclosed by the art cited herein, as well as different variants of these molecules, such as molecules that have at least 70, 80, 90, 95 or 99% homology or sequence identity to the avidin or streptavidin sequences described by the references cited herein, so long as the basic functional binding properties of these molecules for biotin are preserved. Functionally similar avidin or streptavidin sequences may also be identified by being encoded by polynucleotides which crosshybridize under stringent conditions, such as after washing in 0.1×SSC and 0.1% SDS at 68° C. Known avidin and streptavidin polypeptides and polynucleotides are disclosed by the references cited herein and are hereby specifically incorporated by reference.

The term “deglycosylated avidin” refers to a chemically modified form of avidin which has been deglycosylated. An example is Neutravidin™ biotin-binding protein incorporated by reference to Pierce Catalog Number 31000. This term also encompasses immobilized, HP- or AP-conjugated, maleimide-activated forms of Neutravidin™ which are incorporated by reference to the Pierce Catalog (April 2006).

The term “recovered” encompasses separation of one or more constituents of a mixture from the remaining components. Recovery includes selective binding of one component in a mixture to a substrate and its subsequent elution from the substrate after nonbinding components have been removed. Recovery may encompass various degrees of purification including recovery of a>0, 1, 5, 10, 50, 70, 80, 85, 90, 95, 99 or 100% pure product, or alternatively, elimination of >0, 1, 2, 5, 10, 25, 50, 66, 75, 80, 90, 99% of the undesired components of a mixture. These ranges encompass all intermediate subranges and point values.

A “fusion protein” is a protein created through creation of a fusion gene comprising a polynucleotide encoding a first peptide sequence, removing any stop codon, and splicing in-frame a polynucleotide encoding a second or subsequent peptide sequence. The fusion gene can then be expressed in a cell as a single protein. If desired, a linker sequence may be added at the beginning or end of the sequences encoding the first and second (or subsequent) peptides. The linkers may encode an avidin or neutravidin binding peptide sequence of the invention, or a tag, such as a GST protein tag, FLAG peptide or hexa-histidine (His)₆₋₈ peptide which binds to nickel or cobalt. The linker may also encode a cleavage site for a peptidase or a chemical agent that permits a portion of the fusion protein to be removed, for instance, a tag or the avidin or neutravidin binding peptide. In addition to “recombinant fusion proteins” the term “fusion protein” is also intended to encompass chemically engineered protein conjugates, such as those chemically linked together or chemically synthesized.

An “exogenous sequence” is one originating from a different source than the sequence with which it becomes associated. For example, the avidin or Neutravidin binding sequence of the invention would be exogenous to a native or known protein sequence to which it is fused or conjugated. An “endogenous sequence” would be a sequence that forms an integral portion of a native or known protein. Throughout the biological sciences, there is continued interest in developing fast and convenient fusion peptide technologies. To date, these short molecular epitopes have aided tremendously in numerous biochemical studies and promise to facilitate the functional elucidation of uncharacterized gene products [Waugh, Making the most of affinity tags, Trends Biotechnol. 23 (2005) 316-320; Phizicky, et al., Protein-protein interactions: Methods for detection and analysis, Microbiol. Rev. 59 (1995) 94-123]. Yet, while a wide variety of affinity tags have been developed and characterized throughout the years, drawbacks associated with each molecular recognition epitope calls for the continued development of new orthogonal handles. For example, affinity tags derived from antibodies such as the myc-tag or the Flag-tag require denaturing conditions for antibody dissociation [Evan, et al., Isolation of monoclonal antibodies specific for human c-myc proto-oncogene product, Mol. Cell. Biol. 12 (1985) 3610-3616; Brizzard, et al., Immunoaffinity purification of FLAG epitope-tagged bacterial alkaline phosphatase using a novel monoclonal antibody and peptide elution, Biotechniques 16 (1994) 730-735], while whole protein-tags such as glutathione S-transferase or the maltose binding protein often require treatment with site-specific proteases for subsequent biochemical characterization of the desired protein [Nygren, et al., Engineering proteins to facilitate bioprocessing. Trends Biotechnol. 12 (1994) 184-188]. Even affinity tags that have gained widespread use throughout the life sciences can have potential drawbacks such as the His-tag, which can associate with a host of metal chelating contaminants [Essen, et al., Single-step purification of a bacterially expressed antibody Fv fragment by immobilized metal affinity chromatography in the presence of betaine. J. Chromatogr A. 657 (1993) 55-61]. Therefore, the development of new molecular recognition technologies for the isolation, purification and characterization of proteins is of continuing utility. To this end, we have developed the AviD-tag, a NeutrAvidin/avidin specific fusion peptide affinity tag capable of protein immobilization, and purification. Moreover, the orthogonal nature of the AviD-tag and Strep-tag may find numerous applications in cellular labeling technologies [Zhou, et al., Quantum dots and peptides: A bright future together, Peptide Sci. (2007)].

Previous efforts towards the characterization of the phage-display selected motif DXₐAX_{b}PX_{c} (SEQ ID NO: 1) (where Xₐ=R or L; X_{b}=S or T; and X_{c}=Y or W) showed that a high level of selectivity exists for NeutrAvidin/avidin versus streptavidin [Meyer, et al., Highly selective cyclic peptide ligands for NeutrAvidin and avidin identified by phage display, Chem. Biol. Drug Des. 68 (2006) 3-10].

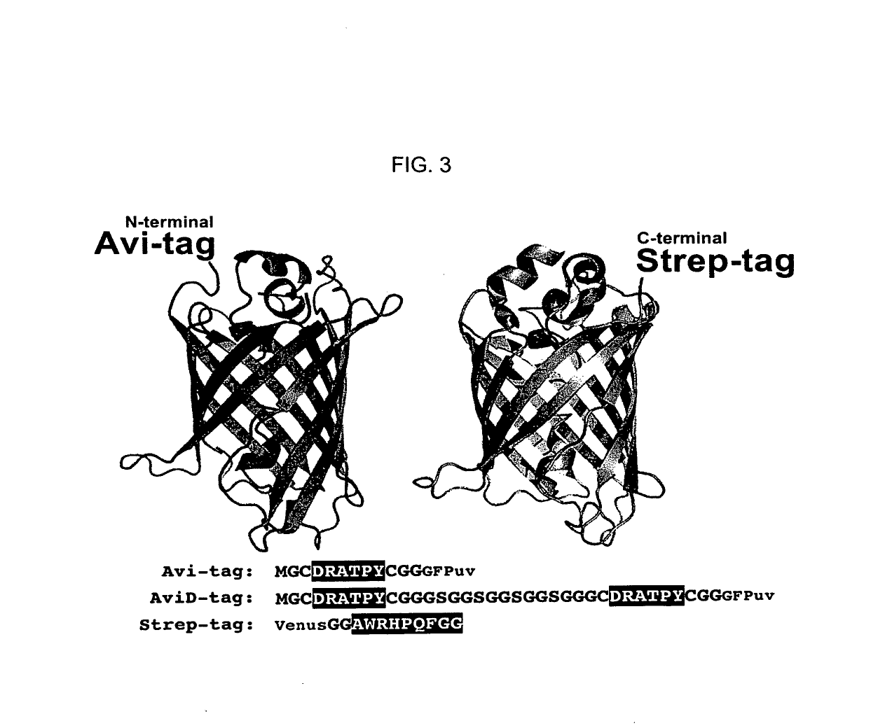

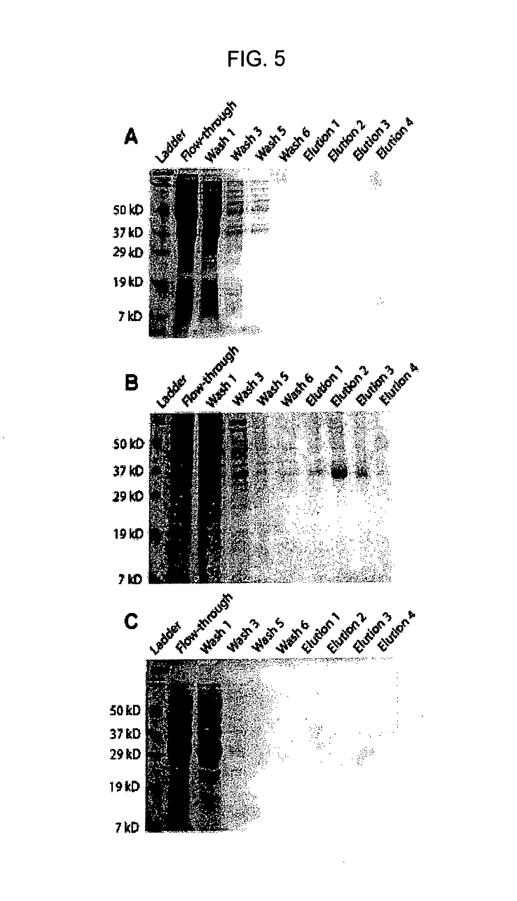

The observed >1000-fold selectivity provides a basis for use of this peptide motif for protein purification and immobilization. Consequently, recombinant fusion proteins containing the DRATPY (SEQ ID NO: 8) moiety were constructed. Using the ultraviolet excitable GFPuv as a model protein, two fusion proteins were produced encoding the DRATPY (SEQ ID NO: 8) peptide: a monovalent variant (Avi-tag) expected to exhibit faster off-rates and lower elution profiles, and a divalent variant (AviD-tag) expected to produce higher levels of protein immobilization and purification as a result of its lower dissociation constant (FIG. 3). The inventors immobilized and eluted the AviD-tag labeled GFPuv onto NeutrAvidin immobilized agarose resin (FIG. 5), in yields greater than Avi-tag labeled GFPuv. To further confirm the necessity of the divalent design, gravity-flow purification against immobilized NeutrAvidin resin from cell lysate was performed with both fusion proteins. Significantly higher levels of protein elution were clearly observed with the divalent affinity tag following affinity purification and subsequent SDS-PAGE analysis (FIG. 5B).

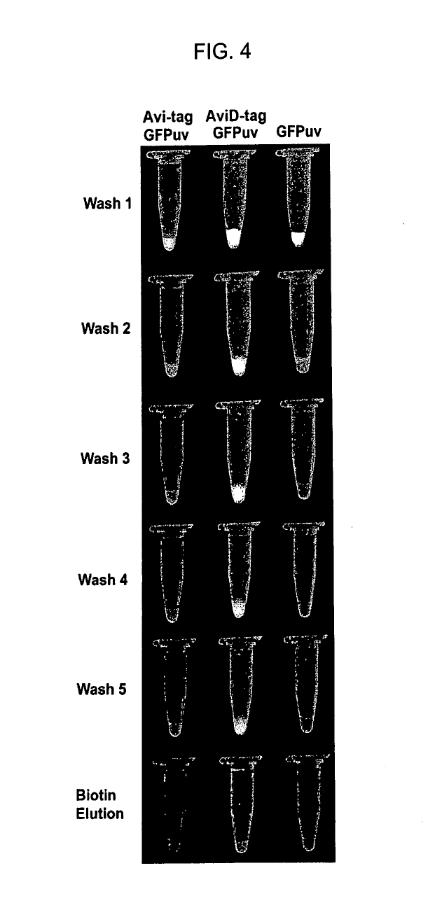

As previously described, members of this phage-display selected motif had been shown to be 1000-fold more selective for NeutrAvidin/avidin versus streptavidin. To demonstrate that the orthogonal behavior of the phage-display selected peptides had successfully translated to the divalent fusion peptide, AviD-tag labeled GFPuv was subjected to gravity-flow purification against streptavidin immobilized agarose resin. The divalent AviD-tag labeled fusion protein was not immobilized by the streptavidin support (FIG. 5C). To further assess the orthogonal nature of the system, a Venus (Yellow Fluorescent Protein variant of GFP) fusion protein containing a C-terminal streptavidin specific Strep-tag, a previously well-characterized affinity tag that binds streptavidin, with a reported dissociation constant of 37 μM, was constructed [Schmidt, et al., Molecular interaction between the Strep-tag affinity peptide and its cognate target, streptavidin, J. Mol. Biol. 255 (1996) 753-766]. This fusion protein was subjected to gravity-flow purification procedures identical to those described for each NeutrAvidin specific fusion protein. Surprisingly, the AviD-tag proved to be more effective than the Strep-tag. Immobilized AviD-tag was shown to be retained at higher levels than that of the Strep-tag and allow for significantly higher levels of protein elution following addition of biotin (FIG. 4). Consequently, the AviD-tag's ability to effectively immobilize proteins under physiological conditions suggests that it will be of value in a host of protein purification and immobilization exercises.

Additionally, the commercial availability of many (strept)avidin conjugates suggests the AviD-tag's utility in a myriad of unique biological applications. While already established as useful tools in protein purification, immobilization, and characterization, genetically fused affinity peptide tags can be utilized for selective biological targeting [Chen, M. et al., Site-specific labeling of cell surface proteins with biophysical probes using biotin ligase, Nat. Methods. 2 (2005) 99-104; Howarth et al., Targeting quantum dots to surface proteins in living cells with biotin ligase, Proc. Natl. Acad. Sci. USA 102 (2005) 7583-7588]. Expressed in conjugation with known receptors, specific fusion peptide motifs can aid in a host of unique cell-surface molecular recognition applications and provide tremendous support for the elucidation of many biochemical phenomena. For example, utilization of peptide motifs that provide orthogonal binding to streptavidin and avidin conjugated to organic fluorophores or quantum dots can provide new and exciting methods for protein detection, imaging and multiplexing. The orthogonal recognition properties of our AviD-tag provide researchers in the biological sciences with a valuable new tool capable of a wide variety of unique cellular applications.

Peptides comprising the motif DXₐAX_{b}PX_{c} containing repeats of this motif, such as dimers, trimers and other oligomers are contemplated, especially those where repeats, dimers, trimers, and oligomers have additively strong dissociation constants, such that the binding constant is equivalent to (K_{d})ⁿ (n=number of repeats of said domain). Thus, two repeats (n=2) of a cyclic DXₐAX_{b}PX_{c} (SEQ ID NO: 1) motif can bind avidin and/or NA with a dissociation constant of <10 nM, assuming a 100 uM K_{d} linear cDXₐAX_{b}PX_{c} (SEQ ID NO: 2, residues 1-7) motif binds streptavidin with a dissociation constant of >500 uM and a cyclic DXₐAX_{b}PX_{c} (SEQ ID NO: 2, residues 1-7) motif does binds streptavidin with a dissociation constant of >100 uM. The linear peptides with sequences incorporating DRATPY (SEQ ID NO: 8) and DRASPY (SEQ ID NO: 6) bind avidin and Neutravidin with dissociation constants less than 500 uM (FIG. 3B). Dimers and oligomers can have additively strong dissociation constants, such that (K_{d})ⁿ (n=number of repeats of said domain). A linear dimer can have dissociation constants of 25 nM. The linear DXₐAX_{b}PX_{c} (SEQ ID NO: 1) motif binds avidin and Neutravidin with a dissociation constant of 100-500 uM. A peptide having a cyclic DXₐAX_{b}PX_{c} (SEQ ID NO: 1) motif can bind to avidin and Neutravidin with a dissociation constant of 500 nM-100 uM.

Peptides comprising DXₐAX_{b}PX_{c} (SEQ ID NO: 1) which lack streptavidin binding sites are also contemplated. For example, peptides comprising DXₐAX_{b}PX_{c} (SEQ ID NO: 1) but which do not contain a Histidine-Proline-Glutamine (HPQ), Histidine-Proline-Methionine (HPM), Histidine-Proline-Asparagine (HPN), or Histidine-Glutamine-Proline (HQP) motif known to bind Streptavidin.

Avidin or avidin-like molecules (e.g., Neutravidin) may be purified from a natural source like chicken egg white or from a synthetic or recombinant source using a peptide comprising the DXₐAX_{b}PX_{c} (SEQ ID NO: 1). If a peptide or cyclic peptide or peptide multimer bearing this motif is immobilized on a chromatography substrate it can be utilized to isolate or purify avidin or avidin-like peptides (e.g., Neutravidin) to which it binds. Specific protocols for such an isolation are incorporated by reference to Vesa et al., “Efficient production of active chicken avidin using a bacterial signal peptide in Escherichia coli”, Biochemical Journal, 2004, 384, 385-390 (Published online Nov. 23, 2004)

Materials and Methods: M13KO7 Helper phage and all enzymes were purchased from New England Biolabs; NeutrAvidin, avidin and streptavidin were obtained from Pierce; peptide synthesis reagents and resin were purchased from Novabiochem; all other reagents, unless otherwise noted, were obtained from Sigma. Library construction. The six residue di-sulfide constrained cyclic peptide library was constructed N-terminal to a peptide linker to the gene III fusion protein encoded by the phagemid vector pCANTAB-5E (Amersham Biosciences). A gene encoding a peptide linker and containing an internal PstI restriction site had been previously cloned into pCANTAB-5E between SfiI and NotI restriction sites to produce pCANTAB-Fos. After transfection into E. coli and subsequent isolation of the amplified pCANTAB-Fos, a gene encoding the cyclic peptide library was cloned into the SfiI and PstI sites of the vector, as previously described (Meyer et al., Biochemistry 2005; 44 (7):2360-8; Rajagopal et al., Bioorg Med Chem Lett 2004; 14 (6):1389-93). The gene was constructed using overlapping primers. The synthesized oligonucleotide library contained the NNS mixed codon set for randomized positions, where N corresponds to G, C, A, or T; and S corresponds to G or C. The primers were obtained from IDT (Integrated DNA Technologies).

LibFwd1:

cgatgcggcccagccggccatgggttgcnnsnnsnnsnnsnnsnnstgcggtggaggc (SEQ ID NO: 25)

LibRev1:

gcaagcgctgcagcaccgcctccaccgca (SEQ ID NO: 26)

The primers were extended to the full duplex DNA by mutually primed synthesis with the Klenow fragment of Escherichia coli DNA polymerase I. The insert was purified and digested with SfiI and Pst I and subsequently ligated into digested pCANTAB-Fos (see supplementary materials incorporated by reference for a complete library sequence). The library was then transformed into XL1-Blue E. coli cells (Stratagene™) via electroporation. The library size was estimated by titration of the transformation mixture on ampicillin- and glucose-containing LB agar plates, and was found to be 1.1×10⁹ CFU. The phagemid DNA from the transformation mixtures was isolated after amplification in E. coli and was re-transformed into new XL1-Blue cells, which were grown overnight with ampicillin and tetracycline selection in the presence of glucose. The library containing E. coli were stored in glycerol (20%) at −78° C.

Phage-displayed peptide selection against NeutrAvidin. XL1-Blue E. coli containing the phagemid library vector were grown from glycerol stocks in 5 mL of 2×YT media with glucose and ampicillin selection at 37° C. Titered M13KO7 helper phage (5×10⁹ pfu) was added when the culture reached an OD₆₀₀ of 0.8 and was incubated for 1 hour. The culture was then pelleted via centrifugation, the cells were resuspended in 2×YT with ampicillin and kanamycin, and allowed to grow overnight. After 10 hours of incubation, the culture was again pelleted by centrifugation and the supernatant was filtered through a 0.45 μm sterile filter to remove trace E. coli. The phage was isolated from the supernatant by PEG (polyethylene glycol) precipitation. 1 mL of 20% PEG in 2.5 M NaCl was added to the 5 mL of filtered media. The resulting precipitate was isolated by centrifugation at 18,000 rcf. The phage pellet was resuspended in 5 mL of Tris Buffer A (20 mM Tris-HCl, 150 mM NaCl, and 0.05% Tween 20 at pH=7.4). The phage was then re-precipitated with 1 mL PEG/NaCl, isolated via centrifugation, and resuspended in 1 mL of Tris Buffer A.

100 μL of the phage solution was then exposed to a well of a NeutrAvidin coated polystyrene plate (Pierce) that had been previously rinsed with Tris Buffer A. After 1 hour of incubation, the phage solution was discarded and the well was washed six times for 10 minutes each with 200 μL of Tris Buffer A. Bound phage was eluted with 200 μL of 0.2 M glycine (pH=2.0) by incubation for 10 minutes, followed by neutralization with 40 μL of 2 M Tris base. The input phage and eluted phage were then used to infect two 5 mL tetracycline-selected cultures of XL1 Blue E. coli (OD₆₀₀=0.8). After 1 hour, the cells were pelleted and resuspended in 5 mL of 2×YT with ampicillin and glucose. To estimate the number of input and output phages, 20 μL of ten-fold serial dilutions of each culture was plated on LB agar plates that contained ampicillin. The rest of the output culture was grown overnight, at which point 1 mL was used to start the next round of selection, while the other 4 mL were stored in glycerol (20%) at −78° C. DNA from colonies from the LB agar plates was isolated for DNA sequencing.

Solid phase peptide synthesis and peptide cyclization. The selected NeutrAvidin-binding peptides were synthesized via standard Fmoc solid-phase peptide synthesis strategy on RinkAmide-AM resin. All peptides were synthesized with an C-terminal glycine and two cysteines flanking the consensus sequences (i.e. CXXXXXXCG) (SEQ ID NO: 3). Cleavage from RinkAmide resin with TFA left an amide bond on the C-terminal carbonyl of the peptide. After cleavage and global deprotection with 94% TFA, 2.5% water, 2.5% ethanedithiol and 1% triisopropylsilane, the peptides were purified essentially as described previously (Zhou et al., Helical supramolecules and fibers utilizing leucine zipper-displaying dendrimers. J Am Chem Soc 2004; 126 (3):734-5). Briefly, the peptides were precipitated three times in chilled ether, and the dried peptides were further purified by HPLC in 0.1% TFA with a gradient of 10%-20% acetonitrile in water. The peptides were lyophilized and either stored at −20° C. for direct use as a linear peptide, or were cyclized before characterization.

Peptide cyclization was carried out by oxidation of the two cysteines to form an intramolecular disulfide bond. The peptides (500 μM) were shaken in PBS (phosphate buffered saline, pH=7.4) with 10% DMSO for 8 hours at 37° C. Extent of the disulfide bond formation was monitored as a loss of free thiol by the DTNB test as reported previously (Zhou, et al., Noncovalent multivalent assembly of jun peptides on a leucine zipper dendrimer displaying fos peptides. Org Lett 2004; 6 (20):3561-4). Reflective phase MALDI mass spectrometry confirmed the peptides' molecular mass, as well as their cyclization states. Results for the cyclized peptides are as follows: DRASPY (SEQ ID NO: 6), expected: 968.0 g/mol, found: 968.4 m/z; DLASPW (SEQ ID NO: 4), expected: 948.0 g/mol, found: 948.0 m/z; DRATPY (SEQ ID NO: 8), expected: 982.1 g/mol, found: 981.8 m/z. Amino acid analysis was also carried out on the cyclized peptides (W.M. Keck Facility, Yale University).

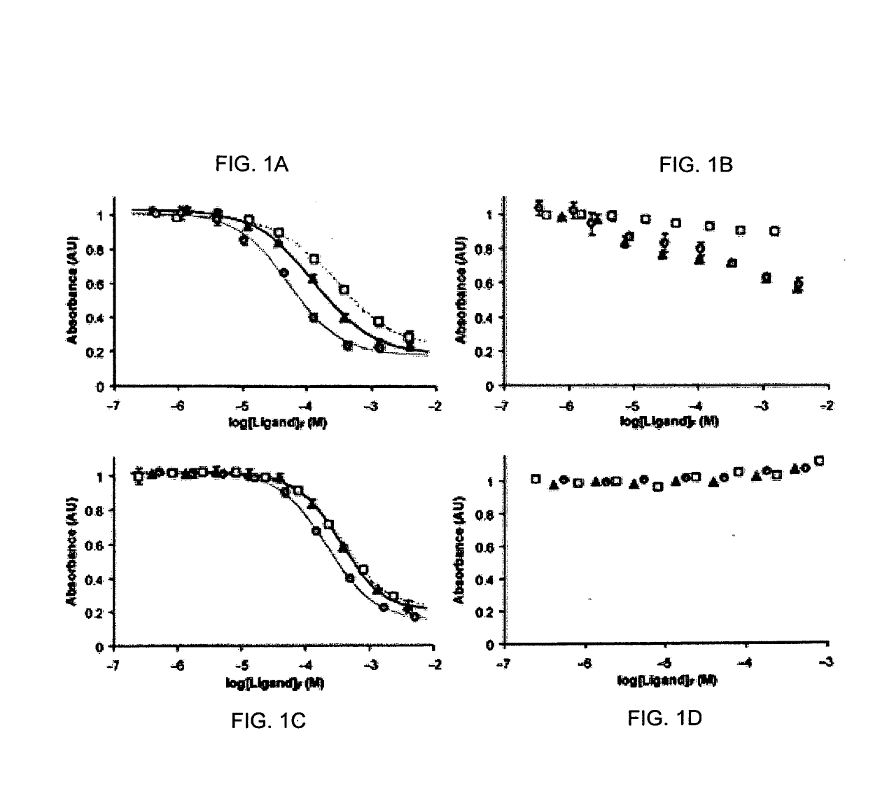

HABA-competitive binding determination. For the competition assays between the NeutrAvidin-selected peptides and HABA, increasing amounts of peptide were titrated into an equimolar complex of HABA and NeutrAvidin, avidin, or streptavidin (50 μM final concentrations) in PBS. After 60 minutes, the absorbance of the complex was monitored at 500 nm. To calculate the IC₅₀s of the selected peptides, the average of three separate trials were fitted to the Hill equation,

$$[ LR ] = {[ L ]}_{B} + \frac{{[ L ]}_{F} - {[ L ]}_{B}}{1 + {( \frac{{IC}_{50}}{[ L ]} )}^{nH}} \qquad ( 1 )$$

where [L] is the total ligand concentration, [L]_{F} is free ligand concentration, [L]_{B} is bound ligand concentration and nH is the Hill coefficient (Hill, The possible effects of the aggregation of the molecules of haemoglobin on its dissociation curves. J Physiol 1910; 40:4-8). Only the NeutrAvidin and avidin data could be fit to equation 1 (FIG. 1). The dissociation constants of the peptides were then determined using,

$${K}_{d} = {K}_{L 2} = \frac{{IC}_{50}}{1 + \frac{[ {L}_{1} ]}{{K}_{L 1}}} \qquad ( 2 )$$

where [L₁] is the HABA concentration, K_{L1} is the dissociation constant of the complex of HABA and the biotin-binding protein, and K_{L2} is the dissociation constant of the selected peptide for the biotin-binding protein (Cheng et al., Relationship between the inhibition constant (K1) and the concentration of inhibitor which causes 50 percent inhibition (150) of an enzymatic reaction. Biochem Pharmacol 1973; 22 (23):3099-108). A calculated value of 15 μM was used for the dissociation constant of the HABA-NeutrAvidin complex (see supplementary material), and the literature value of 7 μM for the HABA-avidin complex (Gree, Spectrophotometric determination of avidin and biotin. Methods Enzymol 1970; 18 (Part 1):418-424). Best fit equations were calculated using KaleidaGraph (Synergy Software).

Biotin-binding proteins are used frequently as immobilization and bioconjugation tools in biotechnology, thus it is important to identify the peptide epitopes that they recognize. In this regard, the most thoroughly studied biotin-binding protein is streptavidin. The early work by Devlin, et al. (Random peptide libraries: a source of specific protein binding molecules. Science 1990; 249 (4967):404-6) found a unique consensus motif, HPQ, which has been confirmed in several later studies (Menendez, et al., The nature of target-unrelated peptides recovered in the screening of phage-displayed random peptide libraries with antibodies. Anal Biochem 2005; 336 (2):145-57). Although most of the studies reported HPQ as a major consensus motif, the flanking sequences varied widely. In the studies done with cyclic peptide libraries, cyclization of the selected peptides led to an increase in binding affinity (Zang et al., Tight-binding streptavidin ligands from a cyclic peptide library. Bioorg Med Chem Lett 1998; 8 (17):2327-32; Giebel, et al., Screening of cyclic peptide phage libraries identifies ligands that bind streptavidin with high affinities. Biochemistry 1995; 34 (47):15430-5). Given the availability and stability of immobilized streptavidin, streptavidin-binding peptides have been used as purification tags (Schmidt et al., Molecular interaction between the Strep-tag affinity peptide and its cognate target, streptavidin. J Mol Biol 1996; 255 (5):753-66; Schmidt et al., The random peptide library-assisted engineering of a C-terminal affinity peptide, useful for the detection and purification of a functional Ig Fv fragment. Protein Eng 1993; 6 (1):109-22) and in other applications (Skerra, et al., Applications of a peptide ligand for streptavidin: the Strep-tag. Biomol Eng 1999; 16 (1-4):79-86). In an effort to reduce the non-specific binding of avidin and streptavidin in such applications, a chemically deglycosylated form of avidin, called NeutrAvidin, was developed. The low cost and low non-specific binding of NeutrAvidin make it an excellent choice for use in immobilization for various applications, especially in vitro selection. However, before selections are carried out, the background binding of the immobilization matrix was characterized.

Phage display selection and synthesis of NeutrAvidin binding peptides. While performing a selection against a separate target, an interesting NeutrAvidin-binding peptide epitope was discovered in the control selections. The rapid and complete convergence to this novel motif encouraged us to repeat the selection against NeutrAvidin and characterize the selected peptides more fully. The selection utilized a six residue cyclic peptide library constrained with a disulfide bond from two conserved cysteines. The library size was chosen to enable complete coverage (1.1×10⁹ unique nucleotide sequences encoding for 6.4×10⁷ unique peptides) and a cyclic architecture was chosen because of the increase in affinity that cyclization provides relative to linear peptides (Giebel, et al., Screening of cyclic peptide phage libraries identifies ligands that bind streptavidin with high affinities. Biochemistry 1995; 34 (47): 15430-5; Fung et al., Design of cyclic and other templates for potent and selective peptide alpha-MSH analogues. Curr Opin Chem Biol 2005; 9 (4):352-8). The library was expressed as a fusion to the gene III protein of M13 filamentous bacteriophage, C-terminal to the periplasmic signaling sequence and N-terminal to a peptide linker and the rest of the gene III protein (see supplementary material). Phage display selection rounds were carried out against NeutrAvidin coated polystyrene plates without further preparation. After only three rounds of selection, a striking motif was discovered (Table 1). The motif is of the general form DXₐAX_{b}PX_{c} (SEQ ID NO: 1), where Xₐ=R or L; X_{b}=S or T; and X_{c}=Y or W. After two more rounds, the selection slightly favored the peptide DRASPY (SEQ ID NO: 6). The consensus sequences all have Asp in the first position, Ala in the third position, and Pro in the fifth position. It is worth noting that even in the positions of variability, close consensus was maintained. For instance, the fourth position strictly requires a hydroxyl containing residue (Ser or Thr) and the sixth position requires an aromatic amino acid (Tyr or Trp). The second position allows the most drastic change within the motif, with Arg as the favored residue, but Leu being tolerated.

TABLE 1
Selected cyclic peptide phage display results

| Round 3 | %^{a} | Round 4 | %^{b} | Round 5 | %^{c} |
|---|---|---|---|---|---|
| CDRASPYC | 27 | CDRASPYC | 46 | CDRASPYC | 49 |
| CDLASPWC | 27 | CDRATPYC | 27 | CDLASPWC | 18 |
| CDRATPYC | 20 | CDLASPWC | 12 | CDRATPYC | 15 |
| CDRASPWC | 20 | CDRASPWC | 8 | CDRASPWC | 5 |

^{a}15 clones sequenced.
^{b}26 clones sequenced.
^{c}40 clones sequenced.

Previous studies targeting streptavidin have found consensus sequences with the motif HPQ (Devlin et al., Random peptide libraries: a source of specific protein binding molecules. Science 1990; 249 (4967):404-6). Other reported streptavidin binding motifs include: GDF/WXF (SEQ ID NO: 12), PWXWL (SEQ ID NO: 13), EPDWF/Y (SEQ ID NO: 11), and DVEAWL/I (SEQ ID NO: 10) (Menendez et al., The nature of target-unrelated peptides recovered in the screening of phage-displayed random peptide libraries with antibodies. Anal Biochem 2005; 336 (2):145-57). It has been shown that HPQ does not bind avidin in its native or deglycosylated state (Kay et al., An M13 phage library displaying random 38-amino-acid peptides as a source of novel sequences with affinity to selected targets. Gene 1993; 128 (1):59-65; Gregory et al., Use of a biomimetic peptide in the design of a competitive binding assay for biotin and biotin analogues. Anal Biochem 2001; 289 (1):82-843), still it is noteworthy that neither this motif, nor any of the minor motifs mentioned above were seen in the selection results. Moreover, the NeutrAvidin-binding epitope that was selected in a multivalent context, VPEY (SEQ ID NO: 14), was not detected (Petrenko et al., Phages from landscape libraries as substitute antibodies. Protein Eng 2000; 13 (8):589-92). The novelty of the selected epitopes, along with their relatively early appearance, led us to further investigate the binding of these peptides to NeutrAvidin. In light of the fact that all of the selected peptides fell into the general motif of DXₐAX_{b}PX_{c}(SEQ ID NO: 1), we decided to synthesize the three most frequently observed peptides for further characterization, namely DRASPY (SEQ ID NO: 6), DLASPW (SEQ ID NO: 4), and DRATPY (SEQ ID NO: 8).

The peptides were synthesized via standard Fmoc strategies and consisted of a C-terminal glycine, the consensus sequence, and two flanking cysteines (CDXₐAX_{b}PX_{c}CG) (SEQ ID NO: 2). All of the consensus peptides readily cyclized upon overnight shaking in PBS with 10% DMSO. The MALDI mass spectrometry confirmed both the monomer status of the peptides, as well as their cyclization state. The composition and concentration of the peptides were confirmed via amino acid analysis.

Competition between HABA and the selected peptides for NeutrAvidin binding. A common method for the quantitation of biotin in solution is a competition assay with the dye HABA, which was developed by Green (Green, A Spectrophotometric Assay for Avidin and Biotin Based on Binding of Dyes by Avidin. Biochem J 1965; 94:23C-24C). The binding of HABA to avidin causes a significant increase in absorbance of light at 500 nm. Since HABA binds avidin in a biotin-competitive manner, it is possible to quantify the amount of biotin in a solution based on the loss of absorbance at 500 nm of the HABA/avidin complex (Green et al., Spectrophotometric determination of avidin and biotin. Methods Enzymol 1970; 18 (Part 1):418-424). As Green noted, the extent to which HABA can be out-competed by a biotin analogue is dependent upon the binding affinity of the competitor). Therefore, we examined NeutrAvidin's HABA-binding ability with the goal of characterizing the selected peptides through a competition assay.

Since the phage display selections against NeutrAvidin did not have any bias towards the biotin-binding site of the protein, it was far from certain that the selected peptides would compete with HABA. However, since a previously discovered HPQ-containing peptide was shown to bind in the biotin/HABA pocket of streptavidin (Katz et al., In crystals of complexes of streptavidin with peptide ligands containing the HPQ sequence the pKa of the peptide histidine is less than 3.0. J Biol Chem 1997; 272 (20): 13220-8), it was believed that the peptides from this selection might bind the analogous site in NeutrAvidin. Therefore, increasing amounts of our selected peptides were titrated into a complex of HABA and NeutrAvidin and monitored the decrease in absorbance at 500 nm (FIG. 1). The decrease in absorbance at 500 nm observed upon addition of the peptide ligands indicates that they are binding in a HABA competitive fashion. Since HABA and biotin are known to bind to the same pocket (Weber et al., Crystallographic and thermodynamic comparison of natural and

TABLE 2
Cyclic peptide binding constants^{a}

| Peptide | NeutrAvidin | Avidin | Streptavidin |
|---|---|---|---|
| 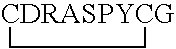 | 31.5 ± 4.4 | 44.9 ± 2.3 | >5,000 |
| 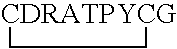 | 12.5 ± 0.7 | 28.1 ± 0.9 | >5,000 |
| 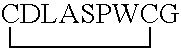 | 62.8 ± 10.8 | 46.2 ± 3.7 | >5,000 |

^{a}K_{d} values are in units of μM

synthetic ligands bound to streptavidin. J Am Chem Soc 1992; 114 (9):3197-3200), it is likely that the selected peptides also bind in the same manner. It is interesting to note that all of the selected peptides assayed for HABA-competitive binding to NeutrAvidin showed affinity, though not in the order of consensus (Table 2). The tightest binder according to the competition assay, DRATPY (SEQ ID NO: 8), was not the major consensus sequence from the selection. These results suggest either a) the selection does not discriminate peptides within a 5-fold affinity variation or b) the extent of cyclization is inconsistent between the peptides on the surface of the phage during the selection.

It was thought that peptides displayed on the surface of phage with two conserved cysteines would spontaneously cyclize under the phage preparation conditions (Barbas et al., Phage Display: A Laboratory Manual. Cold Spring Harbor, N.Y.: Cold Spring Harbor Laboratory Press; 2001). To test the necessity of cyclization for the selected peptides to bind NeutrAvidin, a competition assay was carried out with the uncyclized peptides (FIG. 1). All three peptides that were assayed showed a marked decrease of binding to NeutrAvidin in their uncyclized states. The increase of affinity between receptors and peptides in the cyclized form has been well documented (Zang et al., Tight-binding streptavidin ligands from a cyclic peptide library. Bioorg Med Chem Lett 1998; 8 (17):2327-32; Giebel et al., Screening of cyclic peptide phage libraries identifies ligands that bind streptavidin with high affinities. Biochemistry 1995; 34 (47): 15430-5).

Selectivity of the selected peptides in HABA competitive binding. Having established that the selected peptides indeed bound NeutrAvidin, we wanted to investigate whether or not they could bind avidin (the glycosylated parent of NeutrAvidin) or streptavidin. Therefore, streptavidin and avidin were assayed for peptide binding using the same conditions as the HABA-competitive NeutrAvidin assay (FIG. 1, Table 2). The similarity of the biotin binding pockets of avidin and streptavidin would seem to indicate that peptides binding these sites would not discriminate well between streptavidin and avidin. However, the results show that the NeutrAvidin-selected peptides do not bind streptavidin in a HABA-competitive manner with any measurable affinity (FIG. 1). Indeed, it has been shown that some HPQ-containing peptides that bound streptavidin tightly did not bind avidin (Kay et al., An M13 phage library displaying random 38-amino-acid peptides as a source of novel sequences with affinity to selected targets, Gene 1993; 128 (1):59-65; Gregory et al., Use of a biomimetic peptide in the design of a competitive binding assay for biotin and biotin analogues. Anal Biochem 2001; 289 (1):82-8), indicating that mutually exclusive recognition may be a common theme for these proteins. Avidin, on the other hand, does show significant binding to the NeutrAvidin selected peptides (Table 2). This indicates that the chemical modifications carried out on avidin to produce NeutrAvidin are outweighed in this system by the similarity in primary structure of the two proteins. The similarity in binding constants (Table 2) implies that the binding of the selected peptides is generally independent of the glycosylation state of avidin.

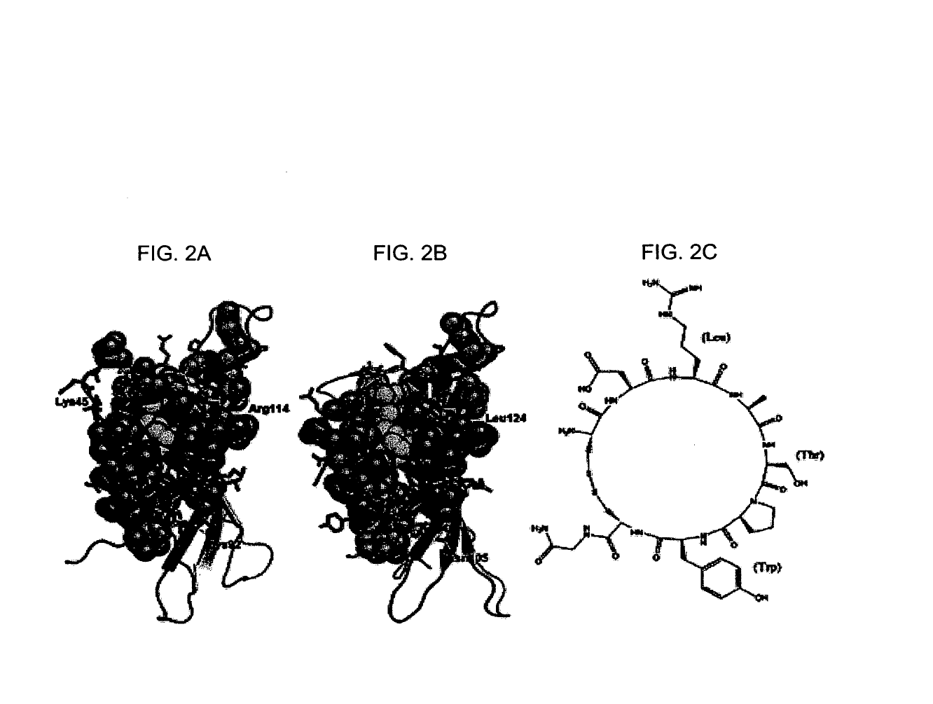

The demonstrated selectivity for avidin and NeutrAvidin is remarkable for the selected peptides, though the DXₐAX_{b}PX_{c} (SEQ ID NO: 1) motif (where Xₐ=R or L; X_{b}=S or T; and X_{c}=Y or W) itself is interesting in a number of ways. First, the Pro that is absolutely conserved at position 5 suggests that the peptides assume a turn motif. Interestingly, the variations seen in positions 4 and 6 are logical substitutions of similar amino acid residues. The appearance of Ser and Thr at position 4 indicate that this residue might be involved in a hydrogen bond, though it is not tightly packed since either residue still binds. Likewise, the allowance of both Trp and Tyr at position 6 indicate some potential π-π interaction. For the absolutely conserved Asp at position 1, it is interesting that no Glu was seen. This might indicate that Asp forms an essential interaction that is very size or distance dependent. The conserved Asp residue might also be the source of specificity for NeutrAvidin vs. streptavidin. If one compares the aligned structures of avidin and streptavidin (FIG. 2) (Pugliese et al., Three-dimensional structure of the tetragonal crystal form of egg-white avidin in its functional complex with biotin at 2.7 A resolution. J Mol Biol 1993; 231 (3):698-710; Weber et al., Structural origins of high-affinity biotin binding to streptavidin. Science 1989; 243 (4887):85-8), the similarity of the binding pocket is striking, considering the two complete proteins only share 33% identity. However, there are two lysines (Lys 45 and Lys92) and two arginines (Arg114 and Arg100) residues near the binding pocket of avidin (within 12 Å of biotin), that are not present in streptavidin. These lysines or arginines could form a salt bridge with the conserved Asp in position 1 of the selected epitopes.

Materials and Methods. NeutrAvidin and streptavidin products were obtained from Pierce. All enzymes were purchased from New England Biolabs. All other reagents, unless otherwise noted, were obtained from Sigma.

Molecular cloning. The plasmids for the Avi-tag conjugates were constructed by cassette mutagenesis in the pET-Duet vector. The Avi-tag cassettes were constructed by extending two overlapping primers with the Klenow fragment of Escherichia coli DNA polymerase I. The primers are as follows:

Avi-tag Forward:

(SEQ ID NO: 27)

5′-GATATACCATGGGCTGCGACAGGGCGACGCCGTACTGCGGTGGGAA
TTCGCTGCAGGG-3′
Avi-tag Reverse:

(SEQ ID NO: 28)

5′-GCATTATGCGGCCGCTTAGTGATGGTGATGGTGATGCAAGCTTCCCT
GCAGCGAATT-3′
AviD-tag Forward:

(SEQ ID NO: 29)

5′-GCAGGACCATGGGCTGCGATCGCGCGACCCCGTATTGCGGCGGTGGA
TCCGGCGGTAGCGGCGGTAGTGG-3′
AviD-tag Reverse:

(SEQ ID NO: 30)

5′-TACAGGGAATTCCCACCGCAATACGGGGTCGCGCGATCGCAGCCACC
GCCGCTACCGCCACTACCGCCGCT-3′

The Avi-tag cassette was cloned into pET-Duet using the NcoI and NotI restriction enzyme sites, and AviD-tag was cloned into the resulting plasmid using the NcoI and EcoRI sites. This resulted in two plasmids, each with an N-terminal Avi-tag and a C-terminal His-tag. The GFPuv gene was isolated from a plasmid obtained from Clonetech by PCR with the following primers:

GFPuv Forward:

(SEQ ID NO: 31)

5′-GCGGTGGGAATTCGAGTAAAGG-3′
GFPuv Reverse:

(SEQ ID NO: 32)

5′-GTGATGCAAGCTTCCCCCTTTGTAGAGCTCATC-3′

The GFPuv insert was cloned into the Avi-tag plasmids between the EcoRI and HindIII restriction enzyme sites using standard protocols to produce pAviGFPuv and pAviDGFPuv. An N-terminal His-tagged fusion of GFPuv that had been previously cloned into pET Duet, pNHTGFPuv, was used as a control construct [Stains, et al., Site specific detection of DNA methylation utilizing mCpG-SEER, J. Am. Chem. Soc. 128 (2006) 9761-9765].

The Venus gene was obtained in a plasmid as a generous gift from Dr. Atsushi Miyawaki (RIKEN Brain Science Institute, Japan) and had been previously cloned into pRSF-Duet with an N-terminal His-tag. A C-terminal Strep-tag was constructed by cloning into the SalI and NotI restriction sites, to form pStrepVenus, using the following complementary primers:

Strep-tag Forward:

(SEQ ID NO: 33)

5′-CGTACAAGGTCGACGGTGGCGCGTGGCGCCATCCGCAGTTTGGCGGC
TAAGCGGCCGCATAATGC-3′
Strep-tag Reverse:

(SEQ ID NO: 34)

5′-GCATTATGCGGCCGCTTAGCCGCCAAACTGCGGATGGCGCCACGCGC
CACCGTCGACCTTGTACG-3′

Protein expression and purification. Identical expression and purification strategies were used for each recombinant fusion protein. pAviGFPuv, pAviDGFPuv, pStrepVenus and pNHTGFPuv were transformed into E. coli BL21 (DE3) via electroporation. Single colonies were picked and grown overnight in 100 mL of 2×YT media with ampicillin at 37° C. with shaking. The overnight culture was used to inoculate 1 L of 2×YT medium with ampicillin at a starting OD₆₀₀ of 0.08. The cells were grown to an OD₆₀₀ of 0.80 before induction with 1 mM isopropyl-β-D-thiogalactopyranosid (IPTG). After 6 hours, the cells were harvested via centrifugation at 4,500 g for 5 min and resuspended in lysis buffer (50 mM NaH₂PO₄, 300 mM NaCl, pH 8). Cells were lysed using standard sonication protocols and subsequently centrifuged at 18,000 g for 40 min. at 7° C. Protein expressed in the soluble fraction was collected. An initial purification was carried out using immobilized metal affinity chromatography (IMAC) in the following manner: The soluble fraction of the cell lysate was incubated with Ni-NTA agarose resin (Qiagen) for 1 hr. after which it was washed and eluted with increasing concentrations of imidazole (10, 20, 50 and 500 mM). Each protein was further purified by gel filtration chromatography with a HiLoad 16/60 Superdex prep grade column attached to an Amersham FPLC system.

Reflective-phase Matrix-Assisted Laser Desorption (MALDI) mass spectrometry confirmed the fusion proteins' molecular masses to within 3% of the actual mw. Results for each protein are as follows: Avi-tag GFPuv: 28,466 m/z (theoretical 29, 215); AviD-tag GFPuv: 30,996 m/z (theoretical 31,158); N-terminal His-tag GFPuv: 26,719 m/z (theoretical 28,134) and Strep-tag Venus: 29,595 m/z (theoretical 30,057). Protein concentration was determined by Trp absorbance at 280 nm following protein denaturation with 6 M Guanidine HCl. All subsequent NeutrAvidin related immobilization and chromatographic steps were performed in phosphate-buffered saline (PBS: 137 mM NaCl, 10 mM Na₂HPO₄, 2 mM KH₂PO₄, pH 7.4) and all subsequent streptavidin related immobilization and chromatographic steps were performed in 100 mM Tris-HCl with 10 mM EDTA, pH=8.0 at 4° C.

Immobilization of AviD-Tag GFPuv against agarose immobilized NeutrAvidin. The GFPuv fusion proteins containing an N-terminal NeutrAvidin/avidin specific peptide affinity tag and a C-terminal His-tag, for preliminary IMAC, were incubated with agarose resin containing immobilized NeutrAvidin™ protein (Pierce) along side the GFPuv control construct. The NeutrAvidin resin was provided as a 6% cross-linked beaded agarose matrix in 50% aqueous slurry with a binding capacity of approximately 50 nmol of biotinylated antibody/mL of immobilized NeutrAvidin protein. For each immobilization assay, approximately 200 pmol of each GFPuv variant was incubated with 100 μL of immobilized NeutrAvidin protein. The mixture was shaken at room temperature for 1 hr. Following incubation, each sample was centrifuged at 8,000 rpm for 3 min and washed with 100 μL buffer (PBS, pH=7.4). Following each wash, the NeutrAvidin resin for both the tagged and untagged GFPuv variants were exposed to UV light for fluorescence visualization. After 5 washes, both the tagged and untagged GFPuv NeutrAvidin slurries were incubated with 250 μM biotin at room temperature for 30 min. with shaking. Following treatment with biotin, the resin was washed with 100 μL buffer and exposed to UV light for GFPuv visualization.

Affinity purification using agarose immobilized NeutrAvidin/streptavidin resin. 400 μL of agarose immobilized NeutrAvidin and streptavidin resin (Pierce) was packed into separate disposable polystrene columns and equilibrated with 3 mL buffer. Both immobilized NeutrAvidin and streptavidin resin are described as having binding capacities of approximately 50 nmol of biotinylated target/mL. Approximately 100 μg of FPLC purified tagged GFPuv and Venus were diluted with 2 mL soluble cell lysate and incubated with a gel packed column at room temperature for 1 hr with shaking (streptavidin based products were incubated at 4° C. with shaking). Following collection of the flow-though, each column was washed with 400 μL buffer. After 6 column washes, immobilized protein was eluted with 500 μM biotin. Each eluate was collected in 400 μL fractions. Levels of protein elution and purity were analyzed by 15% sodium dodecyl sulfate polyacrylamide gel electrophoresis (SDS-PAGE). Protein bands were visualized by staining with Coomassie brilliant blue. Levels of fluorescence intensity for all chromatographic washes and elution's were measured with a Molecular Devices' SpectraMax Gemini microplate spectrofluorometer.

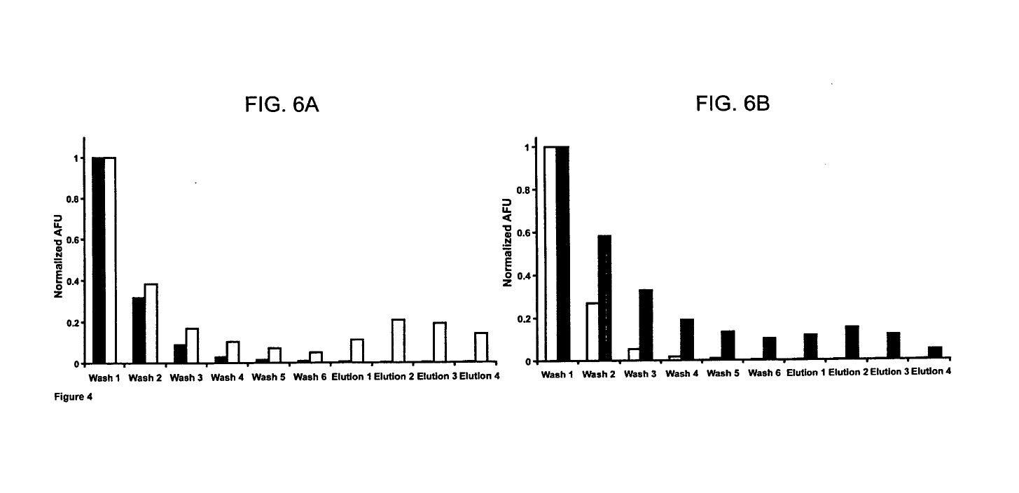

Immobilization and biotin dependent release of Avi-Tag labeled fusion proteins. Recombinant fusion proteins containing the DRATPY (SEQ ID NO:8) moiety were constructed and successfully immobilized onto agarose immobilized NeutrAvidin protein. Two ultraviolet excitable Green Fluorescent Protein (GFPuv) conjugates encoding two variations of the DRATPY (SEQ ID NO: 8) sequence, the Avidin-tag (Avi-tag) and Avidin-Di-tag (AviD-tag) were expressed in E. coli BL-21 alongside the control GFPuv construct (FIG. 3). To assess immobilization levels for each Avi-tag, approximately 6 μg (200 pmol) of each GFP fusion protein was incubated with 100 μL of the NeutrAvidin slurry. Following immobilization, each resin was washed with multiple 100 μL aliquots of buffer and isolated via centrifugation. Following each wash, NeutrAvidin resins incubated with both the tagged and untagged GFPuv variants were photographed under UV light, which enabled direct visualization of GFPuv immobilization (FIG. 6). Following the first wash, fluorescence was seen in both the buffer wash as well as in the NeutrAvidin resin for both the tagged and untagged GFPuv proteins. For the untagged GFPuv variant, minor levels of fluorescence were observed on the NeutrAvidin resin after the second wash and by the third wash, no fluorescence was observed. Fluorescence was observed for the Avi-tag-GFPuv fusion protein, however a stepwise decrease in immobilized protein was witnessed for each subsequent wash. Notably, for the AviD-tag-GFPuv fusion protein, uniform fluorescence intensity was observed for each subsequent wash. Following the successful immobilization of the affinity labeled GFPuv fusion protein, 100 μL of a 250 μM biotin containing solution was incubated with each NeutrAvidin resin for 30 min. at room temperature with shaking. Upon separation of the buffer and resin, a clearly visible decrease in fluorescence was observed for the NeutrAvidin resin immobilized with AviD-tag-GFPuv accompanied by the corresponding appearance of fluorescence in the elution buffer wash (FIG. 4).

Purification of fusion proteins from cell lysate. It was next sought to develop a general strategy for the one step purification of recombinant proteins under gentle conditions. We explored the practicality and applicability of our affinity tag with gravity-flow purification from cell lysate. For each purification exercise, approximately 100 μg of Avi-tag and AviD-tag labeled GFPuv was mixed with 2 mL of prepared E. coli lysate. Each crude cell lysate mixture containing affinity labeled GFPuv protein was then subjected to gravity-flow purification. Following flow-through collection, 400 μL aliquots of buffer were used to wash the column. After six washes, the NeutrAvidin resin was treated with 500 μM biotin and collected in 400 μL fractions. The wash and elution samples were subsequently analyzed by 15% SDS-PAGE. The results confirmed our earlier observations that the designed divalent peptide sequence, AviD-Tag, is the more potent affinity tag. SDS-PAGE analysis suggests the lower affinity of the monovalent Avi-Tag resulted in higher levels of protein elution during the washing procedure (FIG. 5A), while the divalent AviD-Tag was able to withstand rigorous washing and could be successfully eluted upon addition of biotin (FIG. 5B). Further, AviD-tag labeled protein was shown to be largely homogenous, containing no major contaminants.

Demonstration of orthogonality and applicability in comparison to the Strep-tag. Members of the motif DXₐAX_{b}PX_{c} (SEQ ID NO: 1) (where Xₐ=R or L; X_{b}=S or T; and X_{c}=Y or W) have been shown to not bind streptavidin [Meyer, et al., Highly selective cyclic peptide ligands for NeutrAvidin and avidin identified by phage display, Chem. Biol. Drug Des. 68 (2006) 3-10; Krumpe, et al., T7 lytic phage-displayed peptide libraries exhibit less sequence bias than M13 filamentous phage-displayed peptide libraries, Proteomics 6 (2006) 4210-4222], if applied to fusion peptide technologies, peptide discrimination amongst receptors could become a powerful tool for the study of many biological systems. To explore the specificity of AviD-tag for NeutrAvidin, the GFPuv fusion protein was subjected to gravity-flow purification against agarose immobilized streptavidin resin under conditions identical to those of the NeutrAvidin resin. Subsequent SDS-PAGE analysis of buffer washes and biotin elutions revealed the fusion protein was unable to bind to the column, eluting off entirely by the second wash (FIG. 5C). To further demonstrate the potential of an orthogonal peptide based labeling system, a Yellow Fluorescent Protein variant (Venus) containing a streptavidin specific C-terminal Strep-Tag was constructed (FIG. 3). The Venus fusion protein was produced under conditions identical to those previously described. Following purification, approximately 3 nMol of recombinant protein was mixed with cell lysate. The crude cellular mixture was incubated separately with 400 μL of both immobilized NeutrAvidin and streptavidin resin at 4° C. While the Strep-tag fusion protein was successfully immobilized and purified from the streptavidin solid support, it was unable to bind to immobilized NeutrAvidin. In addition to the AviD-tag's orthogonal nature, fusion proteins labeled with the divalent construct were purified in yields greater than that of Strep-tag labeled Venus (FIG. 6A), which was observed to have significantly higher levels of protein dissociate from off the resin during wash procedures.

### INCORPORATION BY REFERENCE

Each document, patent, patent application or patent publication cited by or referred to in this disclosure is incorporated by reference in its entirety. Specifically, the art cited in the Background section teaching particular biological methods or methodologies is incorporated by reference as to its teachings of conventional procedures to which the present invention may be applied. The following supplementary material is incorporated by reference to Meyer et al., Chem. Biol. Drug. Res. 68:3-10 (2006) which is also available online (www.) at blackwell-synergy.com: Table S1: The sequence of the peptide library; FIG. S1: HABA saturation of NeutrAvidin; Table S2: Sequences from the cyclic peptide phage display against NeutrAvidin. No admission is made that any such reference constitutes applicable prior art and the right to challenge the accuracy and pertinence of the cited documents is reserved.

1. An isolated polynucleotide that encodes a polypeptide comprising DXₐAX_{b}PX_{c} (SEQ ID NO: 1).

2. The isolated polynucleotide that encodes a polypeptide comprising DLASPW (SEQ ID NO: 9), DRASPY (SEQ ID NO: 6), or DRATPY (SEQ ID NO: 8).

3. The isolated polynucleotide of claim 1 that encodes a polypeptide comprising (CDXₐAX_{b}PX_{c}CG) (SEQ ID NO: 2).

4. The isolated polynucleotide of claim 1 that encodes a polypeptide comprising CDLASPWCG (SEQ ID NO: 5), CDRASPYCG (SEQ ID NO: 7), or CDRATPYCG (SEQ ID NO: 9).

5. The isolated polynucleotide that encodes a polypeptide comprising DRATPY (SEQ ID NO: 8).

6. The isolated polynucleotide of claim 1, further comprising a polynucleotide sequence that encodes an exogenous polypeptide of interest.

7. A vector or phage comprising the isolated polynucleotide of claim 1.

8. A host cell comprising the vector or phage of claim 7.

9. A method for producing a polypeptide that binds to avidin or Neutravidin comprising:
expressing the polynucleotide of claim 1 in a host cell for a time and under conditions suitable for expression of a polypeptide comprising DXₐAX_{b}PX_{c} (SEQ ID NO: 1) that binds to avidin or Neutravidin; and
recovering said polypeptide.

10. An isolated polypeptide comprising DXₐAX_{b}PX_{c} (SEQ ID NO: 1) or (CDXₐAX_{b}PX_{c}CG) (SEQ ID NO: 2).

11. The isolated polypeptide of claim 10 which comprises DLASPW (SEQ ID NO: 4), DRASPY (SEQ ID NO: 6), DRATPY (SEQ ID NO: 8), CDLASPWCG (SEQ ID NO: 5), CDRASPYCG (SEQ ID NO: 7), or CDRATPYCG (SEQ ID NO: 9).

12. The polypeptide of claim 10 which is cyclic.

13. The polypeptide of claim 10 which is linear.

14. The polypeptide of claim 10 which comprises at least two units of DXₐAX_{b}PX_{c} (SEQ ID NO: 1).

15. The polypeptide of claim 10 which has a dissociation constant for avidin or Neutravidin less than 10 μM.

16. The polypeptide of claim 10 which has a dissociation constant for avidin or Neutravidin less than 100 μM.

17. The polypeptide of claim 10 which has a dissociation constant for avidin or Neutravidin less than 500 μM.

18. The polypeptide of claim 10 which has a dissociation constant for avidin or Neutravidin less than 100 nM.

19. The polypeptide of claim 10 which has a dissociation constant for avidin or Neutravidin less than 10 nM.

20. The polypeptide of claim 10, which does not bind streptavidin.

21. The polypeptide of claim 10, which does not contain a motif for streptavidin binding selected from the group consisting of Histidine-Proline-Glutamine (HPQ), Histidine-Proline-Methionine (HPM), Histidine-Proline-Asparagine (HPN), Histidine-Glutamine-Proline (HQP), DVEAWL/I (SEQ ID NO: 10), EPDWF/Y (SEQ ID NO: 11), GDF/WXF (SEQ ID NO: 12), PWXWL (SEQ ID NO: 13), and VPEY (SEQ ID NO: 14).

22. The polypeptide of claim 10 which comprises an exogenous amino acid sequence of interest, wherein said polypeptide comprising DXaAXbPXc (SEQ ID NO: 1) or (CDXₐAX_{b}PX_{c}CG) (SEQ ID NO: 2) is attached to the N-terminal or C-terminal of the exogenous amino acid sequence of interest.

23. The polypeptide of claim 10 which is bound to a solid support.

24. A composition comprising the polypeptide of claim 10 and a lipid.

25. The composition of claim 24, wherein the lipid is at least one phospholipid selected from the group consisting of phosphatidylcholine (PC), phosphatidylethanolamine (PE), phosphatidylserine (PS) and phosphatidylinositol (PI), and cholesterol.

26. A micelle or lipid bilayer comprising a phospholipid covalently attached to the polypeptide of claim 10.

27. A method for isolating or purifying a protein that binds to DXₐAX_{b}PX_{c} (SEQ ID NO: 1) or (CDXₐAX_{b}PX_{c}CG) (SEQ ID NO: 2) comprising:
contacting a composition comprising a protein of interest with the polypeptide of claim 10 under conditions suitable for binding,
removing unbound molecules in said composition, and
recovering molecule(s) binding to the polypeptide of claim 10.

28. The method of claim 27, wherein the polypeptide of claim 10 is bound to a solid support.

29. The method of claim 27, wherein the protein to be isolated or purified is tagged or conjugated to the peptide or polypeptide of claim 10.

30. A method for identifying a target molecule that binds to DXaAXbPXc (SEQ ID NO: 1) or (CDXₐAX_{b}PX_{c}CG) (SEQ ID NO: 2), comprising:
contacting said target molecule with the polypeptide of claim 10, which may be tagged or labeled.

31. The method of claim 30, which is an immunoassay, flow cytometry, or bioimaging procedure.

32. The method of claim 30, wherein the target molecule is cross-linked avidin or Neutravidin.

33. An orthogonal selection method comprising differentially selecting molecules based on the binding between avidin or Neutravidin and the polypeptide of claim 10; and based on the binding between streptavidin and a molecule comprising a streptavidin binding motif.

34. A method for identifying off-target binding peptides in a procedure using avidin or Neutravidin comprising identifying peptides comprising DXₐAX_{b}PX_{c} (SEQ ID NO: 1) and identifying such peptides as off-target binders.


SEQUENCE LISTING (34 sequences)

SEQ ID NO: 1 - protein, 6 aa, artificial sequence
  synthetic construct
  MISC_FEATURE (2)..(2): X is R or L
  MISC_FEATURE (4)..(4): X is S or T
  MISC_FEATURE (6)..(6): X is Y or W
DXAXPX

SEQ ID NO: 2 - protein, 9 aa, artificial sequence
  synthetic construct
  MISC_FEATURE (3)..(3): X is R or L
  MISC_FEATURE (5)..(5): X is S or T
  MISC_FEATURE (7)..(7): X is Y or W
CDXAXPXCG

SEQ ID NO: 3 - protein, 9 aa, artificial sequence
  synthetic construct
  MISC_FEATURE (2)..(7): X is any amino acid
CXXXXXXCG

SEQ ID NO: 4 - protein, 6 aa, artificial sequence
  synthetic construct
DLASPW

SEQ ID NO: 5 - protein, 9 aa, artificial sequence
  synthetic construct
CDLASPWCG

SEQ ID NO: 6 - protein, 6 aa, artificial sequence
  synthetic construct
DRASPY

SEQ ID NO: 7 - protein, 9 aa, artificial sequence
  synthetic construct
CDRASPYCG

SEQ ID NO: 8 - protein, 6 aa, artificial sequence
  synthetic construct
DRATPY

SEQ ID NO: 9 - protein, 9 aa, artificial sequence
  synthetic construct
CDRATPYCG

SEQ ID NO: 10 - protein, 6 aa, artificial sequence
  synthetic construct
  MISC_FEATURE (6)..(6): X is L or I
DVEAWX

SEQ ID NO: 11 - protein, 5 aa, artificial sequence
  synthetic construct
  MISC_FEATURE (5)..(5): X is F or Y
EPDWX

SEQ ID NO: 12 - protein, 5 aa, artificial sequence
  synthetic construct
  MISC_FEATURE (3)..(3): X is F or W
  MISC_FEATURE (4)..(4): X is any amino acid
GDXXF

SEQ ID NO: 13 - protein, 5 aa, artificial sequence
  synthetic construct
  misc_feature (3)..(3): Xaa can be any naturally occurring amino acid
PWXWL

SEQ ID NO: 14 - protein, 4 aa, artificial sequence
  synthetic construct
VPEY

SEQ ID NO: 15 - protein, 8 aa, artificial sequence
  synthetic construct
CDLASPWC

SEQ ID NO: 16 - protein, 8 aa, artificial sequence
  synthetic construct
CDRASPYC

SEQ ID NO: 17 - protein, 8 aa, artificial sequence
  synthetic construct
CDPASPYC

SEQ ID NO: 18 - protein, 8 aa, artificial sequence
  synthetic construct
CDRAAPWC

SEQ ID NO: 19 - protein, 8 aa, artificial sequence
  synthetic construct
CDRATPYC

SEQ ID NO: 20 - protein, 8 aa, artificial sequence
  synthetic construct
CDRASPWC

SEQ ID NO: 21 - protein, 8 aa, artificial sequence
  synthetic construct
CDWSAPYC

SEQ ID NO: 22 - protein, 8 aa, artificial sequence
  synthetic construct
CDFASPWC

SEQ ID NO: 23 - protein, 8 aa, artificial sequence
  synthetic construct
CDLSTPYC

SEQ ID NO: 24 - protein, 8 aa, artificial sequence
  synthetic construct
CDLSTPYC

SEQ ID NO: 25 - DNA, 58 bp, artificial sequence
  synthetic construct
  misc_feature (29)..(30): n is a, c, g, or t
  misc_feature (32)..(33): n is a, c, g, or t
  misc_feature (35)..(36): n is a, c, g, or t
  misc_feature (38)..(39): n is a, c, g, or t
  misc_feature (41)..(42): n is a, c, g, or t
  misc_feature (44)..(45): n is a, c, g, or t
cgatgcggcccagccggccatgggttgcnnsnnsnnsnnsnnsnnstgcggtggaggc

SEQ ID NO: 26 - DNA, 29 bp, artificial sequence
  synthetic construct
gcaagcgctgcagcaccgcctccaccgca

SEQ ID NO: 27 - DNA, 57 bp, artificial sequence
  synthetic construct
gatataccatgggctgcgacagggcgacgccgtactgcggtgggaattcgctgcagg

SEQ ID NO: 28 - DNA, 57 bp, artificial sequence
  synthetic construct
gcattatgcggccgcttagtgatggtgatggtgatgcaagcttccctgcagcgaatt

SEQ ID NO: 29 - DNA, 70 bp, artificial sequence
  synthetic construct
gcaggaccatgggctgcgatcgcgcgaccccgtattgcggcggtggatccggcggtagcggcggtagtgg

SEQ ID NO: 30 - DNA, 71 bp, artificial sequence
  synthetic construct
tacagggaattcccaccgcaatacggggtcgcgcgatcgcagccaccgccgctaccgccactaccgccgct

SEQ ID NO: 31 - DNA, 22 bp, artificial sequence
  synthetic construct
gcggtgggaattcgagtaaagg

SEQ ID NO: 32 - DNA, 33 bp, artificial sequence
  synthetic construct
gtgatgcaagcttccccctttgtagagctcatc

SEQ ID NO: 33 - DNA, 65 bp, artificial sequence
  synthetic construct
cgtacaaggtcgacggtggcgcgtggcgccatccgcagtttggcggctaagcggccgcataatgc

SEQ ID NO: 34 - DNA, 65 bp, artificial sequence
  synthetic construct
gcattatgcggccgcttagccgccaaactgcggatggcgccacgcgccaccgtcgaccttgtacg